SEQUENCE LISTING (7 sequences)

SEQ ID NO: 1 - protein, 11 aa, unidentified
GWGPLGLARCG

SEQ ID NO: 2 - protein, 12 aa, unidentified
GWGPLGLEEACG

SEQ ID NO: 3 - protein, 9 aa, unidentified
PLGLEEACG

SEQ ID NO: 4 - protein, 7 aa, unidentified
RDVLPGT

SEQ ID NO: 5 - protein, 16 aa, unidentified
RQIKIWFQNRRMKWKK

SEQ ID NO: 6 - protein, 14 aa, unidentified
FCEEFQVPGRGELH

SEQ ID NO: 7 - protein, 16 aa, unidentified
RQIKIWFQNRRMKWKK

(19) United States
(12) Patent Application Publication
(10) Pub. No.: US 2026/0295119 A1
(43) Pub. Date: Oct. 1, 2026

(54) TARGET FOR COMBATTING THE FOREIGN BODY RESPONSE TO IMPLANTABLE BIOMATERIALS

(71) Applicant: The Regents of the University of Colorado, a body corporate, Denver, CO (US)

(72) Inventors: Stephanie Bryant, Boulder, CO (US); Brittany J. Thompson, Longmont, CO (US); Emerson Grey, Broomfield, CO (US)

(73) Assignee: The Regents of the University of Colorado, a body corporate, Denver, CO (US)

(21) Appl. No.: 19/476,690

(22) PCT Filed: Apr. 18, 2024
(86) PCT No.: PCT/US24/25137

§ 371 Date: Oct. 18, 2025

Related U.S. Application Data
(60) Provisional application No. 63/460,176, filed on Apr. 18, 2023.

(51) Int. Cl. A61L 27/34; A61L 27/54

(52) U.S. Cl. CPC A61L 27/34; A61L 27/54; A61L 2300/432; A61L 2300/436; A61L 2300/602; A61L 2300/606

(57) ABSTRACT

Systems and methods to eliminate or reduce the foreign body response (FBR) that occurs when a medical device or other material is implanted into a patient. The FBR causes a chronic inflammatory response that leads to the encapsulation of a medical device by a fibrous capsule. Macrophages have been discovered to become persistent as a result of the implanted biomaterial which occurs by an up-regulation in TLR2 and TLR4. This persistence of macrophages appears to be the primary driver of the FBR. Re-sensitizing macrophages to apoptosis using a small molecule inhibitor of TLR2 and TLR4 will abrogate the formation of the fibrous capsule in the FBR.

### CROSS-REFERENCE TO RELATED APPLICATIONS

This application is the National Stage of International Application No. PCT/US24/25137, filed Apr. 18, 2024, which claims the benefit of U.S. Provisional Application No. 63/460,176 filed Apr. 18, 2023.

### STATEMENT OF GOVERNMENT INTEREST

This invention was made with government support under grant numbers R21 AR071550 and 2R01AR069060-04A1 awarded by the National Institutes of Health. The government has certain rights in the invention.

### REFERENCE TO AN ELECTRONIC SEQUENCE LISTING

The contents of the electronic sequence listing (0112-28-US1-SequenceData.xml; Size: 7,275 bytes; and Date of Creation: Nov. 4, 2025) is herein incorporated by reference in its entirety.

### FIELD OF INVENTION

This invention relates to methods and devices for improving the biocompatibility of implantable medical devices.

### BACKGROUND OF THE INVENTION

The invention addresses the need for improving the biocompatibility of implantable medical devices. While many medical devices can function with a fibrous capsule, this capsule is responsible for implant failure (e.g., implant loosening of joint arthroplasty) and has inhibited advancement of implantable sensors where sensitivity of the sensor depends on direct communication with the host.

### SUMMARY OF THE INVENTION

The present invention provides therapeutic biomaterials that inhibit the foreign body response to non-biological materials, such as orthopedic implants, that are implanted in a living subject, such as a mammal.

The foreign body response (FBR) occurs essentially to all non-biological materials that are implanted into mammals. The FBR is characterized by chronic inflammation that leads to the walling-off of implants by a fibrous capsule, which can lead to implant failure. It is shown herein that Toll-like receptors (TLRs) mediate the FBR and that inhibiting, specifically TLR2 and TLR4, inhibits fibrous capsule formation to several different types of implants.

TLR2 and separately TLR4 either have a role in the FBR depending on the type of material. However, when both TLR2 and TLR4 are mutated in a mouse model, the fibrous capsule is substantially reduced across several different types of implants and to levels that are similar across the materials.

Also demonstrated herein is an enzyme-cleavable delivery system, such as cleavable from a PEG hydrogel, that can release a small molecule therapeutic in response to a stimulant. The delivery system can be used to address chronic inflammation and the FBR through the release of an inhibitor to MyD88 (e.g., inhibitor T6167923), an inhibitor to TRIF, an inhibitor to TLR2 and/or TLR4, or a combination of inhibitors to TLR2, TLR4, TRIF, and/or MyD88. MyD88 was selected and its ability to attenuate pro-inflammatory IL-6 cytokine production in a dose-dependent manner is illustrated herein. A similar approach can be employed with an inhibitor of TRIF, such as through immobilization of the TRIF inhibitor, with or without a MyD88 inhibitor, thus targeting both of the pathways in the TLR4 as needed. A peptide sequence cleavable by MMP-12 was chosen as a linker to facilitate tethering of the inhibitor to a surface of an implantable device because of the MMP-12 enzyme's role in inflammation and disease and its prolific secretion by macrophages. Subcutaneous implantation of the therapeutic PEG hydrogel led to a 50% reduction in inflammatory cell layer thickness relative to control.

We also show here immobilization of the inhibitors outlined herein into a hydrogel. However, a similar approach could be done with any material. As one example, any biomaterial surface could be treated with a silane to produce reactive thiols, acrylates, etc. which can then be used to attach the inhibitor with a linker such as a PEG linker with or without a degradable linker (e.g., MMP12-sensitive used in the attached document) to the surface of an implant. It is contemplated that some inhibitors would not require the linker to be degradable (e.g., TLR2 inhibitor and TLR4 inhibitor). In contrast, MyD88 inhibitors and TRIF inhibitors would preferably include a degradable or cleavable linker.

Inhibitors of TLR4 include M62812, TLR4-IN-C34, and Resatorvid (TAK-242). Further TLR4 inhibitors are listed in Table 1, below. Inhibitors of TLR2 include TLR2-IN-C29, MMG 11, and CU CPT 22. Further TLR2 inhibitors are listed in Table 2, below.

In a first aspect the present invention provides a first coating for an implantable biomaterial. The coating has a small molecule TLR2 inhibitor conjugated to a first linker and a small molecule TLR4 inhibitor conjugated to a second linker. The linkers can have a cleavage site that is cleavable by macrophage elastase. The linkers facilitate immobilization of the small molecule inhibitors on the surface of an implantable biomaterial.

In certain embodiments the coating of the first aspect can also have a cFLIP inhibitor conjugated to a linker. The linker facilitates immobilization of the cFLIP inhibitor on the surface of an implantable biomaterial. In an advantageous embodiment, the cFLIP inhibitor is YM155. It is further contemplated that the cFLIP inhibitor is a cFLIP inhibitor selected from Table 4. The coating according to the first aspect can include an implantable biomaterial where the small molecule inhibitors with the linkers are releasably affixed to the surface of the implantable biomaterial. The small molecule inhibitors with the linkers can be releasably affixed to the surface of the implantable biomaterial using an oxygen plasma/silanization method.

In advantageous embodiments the coating of the first aspect, the TLR2 inhibitor is MMG11 and/or TLR2-IN-C29. It is further contemplated that the TLR2 inhibitor is an inhibitor listed in Table 2.

In further advantageous embodiments the coating of the first aspect, the TLR4 inhibitor is TAK 242, TLR4-IN-C34, and/or M62812. It is further contemplated that the TLR4 inhibitor is an inhibitor listed in Table 1.

As discussed above, the linkers can have a cleavage site that is cleavable by macrophage elastase. In an advantageous embodiment the cleavage site that is cleavable by macrophage elastase has a sequence that is recognizable/cleavable by an MMP12 enzyme. It is further contemplated that the cleavage site sequence is 70%, 80%, or 90% identical to SEQ ID NO: 1, SEQ ID NO: 2 or SEQ ID NO: 3. These sequences are disclosed below. In still further embodiments, the cleavage site sequence is identical to SEQ ID NO: 1, SEQ ID NO: 2 or SEQ ID NO: 3.

In a more general sense, the linker according to the first aspect is a peptide-tether that is sensitive to enzymes that catalyze the hydrolysis of peptidic bonds and the enzymes are overexpressed responsive to inflammation. The enzymes can be matrix metalloproteinases (MMPs).

As discussed above, the coating includes a TLR2 inhibitor, a TLR4 inhibitor and/or a cFLIP inhibitor. The TLR2 inhibitor, the TLR4 inhibitor and/or the cFLIP inhibitor can be custom synthesized to have a sulfhydryl group to facilitate conjugation to the linker.

An implantable biomaterial is further contemplated. The biomaterial can have a surface of poly(ether ether ketone) (PEEK) medical grade silicone (MGS) or poly(ethylene glycol) (PEG), among others. The surface coating can be a surface coating as described above for the first aspect.

In a second aspect the present invention provides a second coating for an implantable biomaterial. The coating according to the second aspect has a TLR2 inhibitor, a TLR4 inhibitor, and an encapsulating agent or hydrogel. The inhibitors can be conjugated to the encapsulating agent or hydrogel. The encapsulating agent or hydrogel can then enable the releasable immobilization of the inhibitor on the surface of an implantable medical device. In certain embodiments the TLR2 inhibitor and the TLR4 inhibitor are conjugated to the encapsulating agent.

In advantageous embodiments the coating of the second aspect, the TLR2 inhibitor is MMG11 and/or TLR2-IN-C29. It is further contemplated that the TLR2 inhibitor is an inhibitor listed in Table 2.

In further advantageous embodiments the coating of the second aspect, the TLR4 inhibitor is TAK 242, TLR4-IN-C34, and/or M62812. It is further contemplated that the TLR4 inhibitor is an inhibitor listed in Table 1.

In certain embodiments the coating of the second aspect can also have a cFLIP inhibitor. In an advantageous embodiment, the cFLIP inhibitor is YM155. It is further contemplated that the cFLIP inhibitor is a cFLIP inhibitor selected from Table 4. The coating according to the second aspect can include an implantable biomaterial where the small molecule inhibitors with the encapsulating agent or hydrogel are releasably affixed to the surface of the implantable biomaterial. The small molecule inhibitors with the encapsulating agent or hydrogel can be releasably affixed to the surface of the implantable biomaterial using an oxygen plasma/silanization method. The systems and methods of the present invention are particularly suited to an implantable biomaterial having a surface of poly(ether ether ketone) (PEEK), medical grade silicone (MGS) or poly(ethylene glycol) (PEG), such as when using the coatings according to the second aspect. It is contemplated that the encapsulating agent is a polymer that controls release of the agent. It is contemplated that the agent could be tethered directly into the coating through a functional group such as an acrylate, thiol, norbornene, maleimide, etc. The coating according to the second aspect can be used in a method of coating the medical device or implantable biomaterial, thereby reducing the FBR to that medical device or implantable biomaterial upon implantation in a subject. In an advantageous embodiment, the method of coating the device or biomaterial will be employed to treat a medical device implantable biomaterial that has a surface such as PEEK, MGS, and PEG (e.g., PEG hydrogels formed from a thiol-norbornene step-growth reaction like PEG10 and PEG20 or from PEG multiacrylate or multimethacrylate monomers that are polymerized in a chain growth reaction like PEG-DA).

In a third aspect the present invention provides a method of treating or preventing FBR in a subject having received an implantable biomaterial. The method includes the step of delivering to the site of implantation an inhibitor of TLR2 and an inhibitor of TLR4. In an advantageous embodiment the TLR2 inhibitor is MMG11 or TLR2-IN-C29. It is further contemplated that the TLR2 inhibitor can be an inhibitor listed in Table 2. In an advantageous embodiment the TLR4 inhibitor is TAK 242, TLR4-IN-C34, or M62812. It is further contemplated that the TLR4 inhibitor is an inhibitor listed in Table 1.

The method according to the third aspect can further include the step of delivering a cFLIP inhibitor, such as YM155, to the site of implantation.

In the various aspects of the invention, the coating for the implantable biomaterial can include a small molecule inhibitor of survivin, such as YM155, FL118, SF002-96-1, Terameprocol, WM-127, GDP366, Abbot 8, LLP3, LLP9, S12, Indinavir, Nelfinavir, LQZ-7, LQZ-7F, LQZ-7I, Shepherdin, AICAR, Deazaflavin analog compound 1, UC-112, MX-106, Compound 12b, Compound 10f, Compound 10h, Compound 10k, Compound 10n, PZ-6-QN or combinations of the aforementioned inhibitors. The survivin inhibitors can be conjugated to a degradable linker, wherein the degradable linker has a cleavage site that is cleavable by macrophage elastase and the linker facilitate immobilization of the small molecule inhibitors on the surface of an implantable biomaterial. In further aspects it is contemplated that the coatings taught herein can be used in methods of coating medical devices and other implantable biomaterials.

In a fourth aspect the present invention provides a third coating for an implantable biomaterial. The coating according to the fourth aspect has an inhibitor of cFLIP conjugated to a degradable linker, where the degradable linker has a cleavage site that is cleavable by macrophage elastase. The linker facilitates immobilization of the inhibitor on the surface of an implantable biomaterial. In an advantageous embodiment inhibitor of cFLIP is YM155 or an analog thereof.

In a fifth aspect the present invention provides a method of inhibiting or reducing the FBR responsive to the implantation of a medical device or other implantable biomaterial. The method can include the step of delivering to the site of implantation the small molecule inhibitor YM155 conjugated to a degradable linker. The degradable linker can be a cleavage site that is cleavable by macrophage elastase and the linker facilitates immobilization of the small molecule inhibitor on the surface of an implantable biomaterial. The small molecule inhibitor can be encapsulated in a hydrogel to which the linker is conjugated.

In a sixth aspect the present invention provides a method of inhibiting the foreign body response to a foreign body in a subject. The method includes the step of delivering to the site of the FBR a first inhibitor that inhibits the activity TLR2 and a second inhibitor that inhibits the activity of TLR4. The method will often be performed in a subject receiving an implantable biomaterial and the method is performed to treat or prevent the FBR is responsive to the implantation of one or more implantable biomaterials. The inhibitor is advantageously a small molecule inhibitor TLR2 and/or TLR4, such as the TLR2 and TLR4 inhibitors referred to herein.

In a seventh aspect the present invention provides a method of in vivo testing a biomaterial for TLR2 and TLR4 FBR. The method includes the step of implanting the biomaterial in Wt, TLR2^{-/-}, TLR4^{-/-}, and/or double knockout (TLR2^{-/-}TLR4^{-/-} or DKO) mice and measuring the resultant encapsulation of the biomaterial in each mouse model.

In an eighth aspect the present invention provides a method of in vitro testing a biomaterial for TLR2 and TLR4 FBR. The method includes the steps of isolating macrophages from Wt, single KO (i.e., TLR2^{-/-} or TLR4^{-/-}), and/or DKO mouse strains, seeding the isolated macrophages on a biomaterial to be tested, priming the macrophage with a pro-inflammatory stimulant, and measuring the production of one or more cytokines. The biomaterial can be pre-treated with plasma. In an advantageous embodiment the cytokines screened for will be IL-6, TNF-alpha, and/or IL-18. The pro-inflammatory stimulant can be LPS and/or PAM3CSK4.

In a ninth aspect the present invention provides a coating for an implantable biomaterial, where the coating has a myeloid differentiation primary response protein 88 (MyD88) inhibitor conjugated to a degradable linker. The degradable linker is cleavable by macrophage elastase. The linker facilitates immobilization the inhibitor on the surface of an implantable biomaterial. The degradable linker can include a PEG linker linking the inhibitor to the degradable linker. In an advantageous embodiment the myeloid differentiation primary response protein 88 (MyD88) inhibitor is T6167923. It is contemplated that the myeloid differentiation primary response protein 88 (MyD88) inhibitor can be an inhibitor listed in Table 3. The myeloid differentiation primary response protein 88 (MyD88) inhibitor can be custom synthesized to have a sulfhydryl group, which facilitates conjugation of the inhibitor to the linker.

In certain embodiments according to the ninth aspect the implantable biomaterial will have a hydrogel component, where the linker is a peptide linker that is affixed or conjugated to the hydrogel. The coating for an implantable biomaterial according to the ninth aspect can be part of an implantable biomaterial having a PEEK, MGS, and PEG component surface. The coating is applied to a surface of the implantable biomaterial, such as a biomaterial having a PEEK, MGS, and PEG (e.g., PEG10 or PEG20) surface.

In a tenth aspect the present invention provides a coating for an implantable biomaterial having a TRIF inhibitor conjugated to a degradable linker. The degradable linker is cleavable by macrophage elastase. The linker facilitates immobilization the inhibitor on the surface of an implantable biomaterial. The degradable linker can include a PEG linker linking the inhibitor to the degradable linker. In an advantageous embodiment the TRIF inhibitor is Pepinh-TRIF or resatorvid (TAK 242). The TRIF inhibitor can be custom synthesized to have a sulfhydryl group, which facilitates conjugation of the inhibitor to the linker. The coating for an implantable biomaterial according to the tenth aspect can further include a myeloid differentiation primary response protein 88 (MyD88) inhibitor conjugated to a degradable linker, such as described for the ninth aspect.

In an eleventh aspect the present invention provides a coating for an implantable biomaterial comprising T6167923 conjugated to a MMP cleavable linker, where the linker is cleavable by macrophage elastase. The linker facilitates immobilization small molecule inhibitors, such as T6167923, on the surface of an implantable biomaterial. The T6167923 can be modified to contain a sulfhydryl group to facilitate conjugation to the linker. In an advantageous embodiment the cleavage site that is cleavable by macrophage elastase has an amino acid sequence that is cleavable by an MMP12 enzyme. It is contemplated that the cleavage site can have an amino acid sequence that is 70%, 80%, or 90% identical to SEQ ID NO: 1, SEQ ID NO: 2 or SEQ ID NO: 3, as disclosed below. Advantageously, the cleavage site is identical to SEQ ID NO: 1, SEQ ID NO: 2 or SEQ ID NO: 3.

In a twelfth aspect the present invention provides a coating for an implantable biomaterial having an inhibitor conjugated to a cleavable or degradable linker. The inhibitor can be a myeloid differentiation primary response protein 88 (MyD88) inhibitor or a TRIF inhibitor (or any of the other inhibitors disclosed herein). The linker facilitates immobilization the inhibitor on the surface of an implantable biomaterial. The linker can be a peptide-tether that is sensitive to enzymes that catalyze the hydrolysis of peptidic bonds. It is contemplated that the enzymes are advantageously enzymes that overexpressed responsive to inflammation. In this manner, the release of the inhibitor is targeted to those instances where there is FBR or the like.

In a thirteenth aspect the present invention provides a coating for an implantable biomaterial having a TLR2 inhibitor, a TLR4 inhibitor and/or a cFLIP inhibitor conjugated to a cleavable or degradable linker. The degradable linker facilitates immobilization of small molecule inhibitors on the surface of an implantable biomaterial. The peptide-tether can have a sequence that is recognized by enzymes that catalyze the hydrolysis of peptidic bonds, where the enzymes are enzymes that are overexpressed responsive to inflammation.

The present invention can be utilized in multiple applications related to medical devices, e.g., knee and hip implants, implantable glucose monitors, and implantable biosensors.

### BRIEF DESCRIPTION OF THE DRAWINGS

For a fuller understanding of the invention, reference should be made to the following detailed description, taken in connection with the accompanying drawings, in which:

FIG. 1 is a set of schematics (A)-(B) showing the proposed role of TLR2 and TLR4 signaling in the FBR through surface-adsorbed damaged associated molecular patterns (DAMPs). (A) Schematic of the FBR over time. (B) Simplified signaling pathway of TLR2 and TLR4 activation by DAMPs, inflammasome activation, and production and amplification of IL-18.

FIG. 2 is a drawing with a schematic (A) and four graphs (B)-(E) showing a comparison of biomaterial chemistry and material properties for select biomaterials as utilized herein. (A) Chemical structure of the biomaterials. PEG hydrogels were formed by polymerization of 8-arm PEG-norbornene and PEG dithiol. Schematic of hydrogel crosslinking when formed with different PEG-norbornene concentrations (10 to 20 wt %). (B) Water contact angle measurements of PEEK and MGS. The dashed line indicates transition to hydrophobic from hydrophilic. (C) Mass swelling ratio of PEG10 and PEG20. (D) Compressive modulus measurements across the materials. Data presented on the log scale. (E) Quantification of absorbed plasma proteins on the surface of each biomaterial. Error bars, mean (SD) of 6 to 9 replicates. P≤0.05: #compared to PEEK, % compared to MGS, @ compared to PEG20, bar indicates significance between groups. ‘NSD’ indicates no significant differences between groups. Chemical structures made using ChemDraw.

FIG. 3 is a drawing with a set of schematics (A), a set of images (B) and five graphs (C)-(G) showing the characterization of neutrophil activation at the surface of biomaterials via surface-adsorbed plasma and the role of TLR2 and TLR4. (A) Schematic of in vitro study design: plasma was pre-adsorbed to the surface of each biomaterial. Bone marrow derived neutrophils were seeded on biomaterials with surface-adsorbed plasma and cultured for 6h. (B) Representative confocal microscopy images of neutrophil attachment on each biomaterial surface with pre-adsorbed plasma proteins, F-actin-green and nuclei-blue (scale bar 10 μm). White arrows indicate possible trails of DNA extruded from the neutrophils. Quantification of Wt neutrophils for (C) cell number, (D) neutrophil elastase (NE) production, and (E) myeloperoxidase (MPO) production at 6 h. Comparison of (F) neutrophil elastase (NE) production and (G) myeloperoxidase (MPO) production of Wt, TLR2^{-/-}, TLR4^{-/-}, and DKO neutrophils at 6 h. Data are mean with standard deviation as error bars for 4-6 replicates. P≤0.05: #compared to PEEK, % compared to MGS, & compared to PEG10, @ compared to PEG20, and bars compare between groups. In (F) and (G) brackets represent p≤0.05 comparing between strains within each material.

FIG. 4 is a drawing with a schematic (A), a set of three graphs ((B), (C), (E)), and a set of images (D) showing the characterization of macrophage activation at the surface of biomaterials. (A) Schematic of study design. Peritoneal macrophages were isolated, treated or not with 0.5 ng/ml LPS and 0.5 ng/ml PAM3CSK and seeded on biomaterials without or with surface-adsorbed plasma. (B) IL-6, TNF-α, and IL-18 secretion of macrophages seeded on each biomaterial without pre-adsorbed plasma proteins or LPS/PAM3CSK4. (C) Impact of pre-absorbed plasma proteins on IL-6 and TNF-α secretion of macrophages seeded on each biomaterial with LPS/PAM3CSK4. (D) Representative confocal microscopy images of the impact of pre-adsorbed plasma proteins on macrophage attachment on each biomaterial with pre-activation; F-actin-green and nuclei-blue (scale bar 10 μm (lower magnification) and 2 μm (higher magnification). (E) Impact of LPS/PAM3CSK4 on IL-6, TNF-α, and IL-18 secretion of macrophages seeded on each biomaterial with pre-adsorbed plasma proteins. Data in (B), (C), and (E) are mean with standard deviation as error bars for 4-6 biological replicates. P≤0.05: #compared to PEEK, % compared to MGS, and bars compare between groups at each end.

FIG. 5 is a drawing with a schematic (A) and set of three mixed schematics/graphs (B)-(D) showing the identification of the role of TLR2 and TLR4 in macrophage activation by biomaterials with surface-adsorbed plasma. (A) Schematic of study design. Peritoneal macrophages were isolated, treated with 1 ng/ml LPS, 1 ng/ml PAM3CSK, or with 0.5 ng/ml LPS and 0.5 ng/ml PAM3CSK and seeded on biomaterials with surface-adsorbed plasma. (B) Comparison of IL-6, TNF-α, and IL-18 secretion on Wt vs. TLR2^{-/-} macrophages, treated with LPS. (C) Comparison of IL-6, TNF-α, and IL-18 secretion on Wt vs. TLR4^{-/-} macrophages, treated with PAM3CSK4. (D) Comparison of IL-6, TNF-α, and IL-18 secretion on Wt vs. DKO macrophages, treated with LPS and PAM3CSK4. Data are mean with standard deviation as error bars for 4-6 replicates. P<0.05: #compared to PEEK, % compared to MGS, @ compared to PEG20, and bars compare between groups at each end.

FIG. 6 is a drawing with a schematic (A), two sets of images ((B) and (D)) and a graph (C) showing the characterization of the FBR in Wildtype (Wt) mice. (A) Schematic of subcutaneous implantation mouse model. Four pockets were created in each mouse and different biomaterials were placed in each. (B) Representative Gömöri trichrome images of the FBR to each biomaterial. An asterisk indicates the location of the implant, triangles indicate the ICL, arrows indicate the FC, scale bars are 20 μm. (C) Quantification of trichrome images shown by a stacked bar chart. ICL (top) indicated in red, FC (bottom) in blue. Data are mean with standard deviation as error bars for 6-9 biological replicates. P≤0.05: #compared to PEEK, % compared to MGS, @ compared to PEG20, bar indicated significance between groups. (D) Representative microscopy images to assess the cellular composition of the ICL. Top and middle rows are H&E stained sections (scale bar 100 μm) where cytoplasm is pink and nuclei are purple. Neutrophils were identified by their lobed nucleus (black arrows) and a close-up image is shown in the upper lefthand corner of the image. Bottom row are stained for Mac-3 (brown) to identify macrophages (scale bar 20 μm). Black star indicates location of implant.

FIG. 7 is a drawing with a three sets of images (A)-(C), each set of images with a graph, and a graph (D) showing the characterization of the FBR in TLR knockout mice. Representative Gömöri trichrome images of the FBR and corresponding quantification of the ICL and FC to (A) PEEK, (B) MGS, (C) PEG10, and (D) PEG20 in TLR2^{-/-}, TLR4^{-/-}, and DKO mice. In the images, an asterisk indicates the location of the implant, triangles indicate the ICL, arrows indicate the FC, scale bars are 20 μm. A stacked box plot shows quantification of the ICL (top of each bar) and FC (bottom of each bar) across the three knockout mice and comparing to Wt mice from FIG. 3 (E) A stacked box plot compares the ICL (top) and FC (bottom) to each biomaterial in the DKO strain. Data are mean with error bars as standard deviation for 5-9 biological replicates. P≤0.05: ‘W’ compared to Wt, ‘D’ compared to DKO, and % compared to MGS.

FIG. 8 is a drawing having a graph showing the gene expression of macrophages post isolation from intraperitoneal space of Wt mice. Quantitative real-time polymerase chain reaction data for gene expression of Tnfa and Arg1, indicating an M1 or M2 polarization state respectively. 0 days indicates immediately after isolation, then the macrophages were cultured in 96 well plates for 1 or 2 days and lysed for PCR. Data are mean with standard deviation as error bars for 4-5 replicates. Bars indicate p≤0.05 between genes.

FIG. 9 is a drawing having two graphs showing IL-18 production of Wt macrophages on each biomaterial with pre-adsorbed plasma proteins in response to the addition of pro-inflammatory stimulant. Data are mean with standard deviation as error bars for 4-6 replicates. P≤0.05: #compared to PEEK, and % compared to MGS.

FIG. 10 is a drawing having two graphs showing a comparison of the cytokine production of Wt macrophages across stimulant conditions on the surface of each biomaterial with pre-adsorbed plasma proteins. Bars indicate p≤0.05, comparing priming agents at each end of the bar, within each cytokine and biomaterial.

FIG. 11 is a drawing having a set of three schematics (A)-(C) addressing therapeutic biomaterials to attenuate inflammation. (A) Therapeutic biomaterials were examined for their ability to release an anti-inflammatory payload in response to an external stimulus. (B) A hydrogel delivering a small molecule via a matrix-metalloproteinase cleavable peptide linker to reduce inflammation in the foreign body response (FBR) was chosen as an experimental approach. In this work, the anti-inflammatory payload is a small molecule T6167923 that inhibits the myeloid differentiation primary response gene 88 (MyD88) adaptor protein. (C) MyD88 is an adaptor protein involved in the signaling of most Toll-like receptors (TLRs) and interleukin-1 receptor (IL-1R), which bind pathogen-associated molecular patterns (PAMPs) and damage-associated molecular patterns (DAMPs) to induce NF-κB mediated transcription of pro-inflammatory cytokines.

FIG. 12 is a drawing having a schematic (A) and two graphs ((B) and (C)) addressing the effect of small molecule MyD88 inhibitor and its thiol-derivative on macrophage activation. (A) Schematic of study design. Interleukin-6 (IL-6) production by RAW 264.7 macrophages stimulated with either the commercially available (B) T6167923 (Br) or custom (C) T6167923 (SH) and then exposed to TLR2 agonist PAM3CSK4. Data presented as means with standard deviations as error bars. A one-way ANOVA was performed with § compares to unstimulated cells with no inhibitor treatment; & compares to PAM3CSK4-stimulated cells with no inhibitor treatment; one symbol: p<0.05; two symbols: p<0.01; three symbols: p<0.001.

FIG. 13 is a drawing having a schematic (A) and two graphs (B) addressing the characterization of T6167923 (SH) with Linker. (A) Synthesis of a model degradation product of the inhibitor. Bromoacetamido-dPEG12-TFP ester was reacted with T6167923 (SH) via the bromoacetamido and then with triglycine via the TFP ester. (B) Characterization of the first reaction by monitoring the consumption of inhibitor free sulfhydryl groups by Ellman's assay. Characterization of the second reaction by monitoring the free amines on the triglycine N-termini with Fluoraldehyde assay.

FIG. 14 is a drawing having three graphs ((A), (B), and (E)) and two schematics ((C) and (D)) addressing the characterization of release of inhibitor by MMP-12. MMP-12 secretion by RAW 264.7 macrophages in (A) serum-free chemically-defined (1% ITS) medium and (B) serum-containing (10% fetal bovine serum) medium was assessed by ELISA. (C) Schematic of the synthesis of an acrylate monomer with an MMP-12 sensitive peptide containing a tryptophan residue. The acrylate monomer was reacted with PEG diacrylate to form a hydrogel with MMP-12 sensitive tethers. (D) Schematic of the study design to characterize release by MMP-12 enzyme. (E) Tryptophan cumulative release (%) after MMP-12 cleavage in buffer containing 5 nM activated MMP-12 measured by spectroscopy at 285 nm. Data presented as means with standard deviations as error bars. A one-way ANOVA was performed with a Tukey's post-hoc analysis: Symbols over bars compare to blank condition in panels (A) and (B). Pairwise comparisons represented by symbols over lines. p<0.05 designated by *, p<0.01 by * and p<0.001 by *

FIG. 15 is a drawing having two schematics ((A) and (B)), two images (C), and a graph (D) addressing the therapeutic PEG hydrogel synthesis, RAW 264.7 studies, and subcutaneous implantation. Inhibitor-dPEG12-peptide was synthesized, then conjugated to acrylate-PEG-maleimide (molecular weight=3.4 kDa). The product was combined with PEGDA (molecular weight=3.4 kDa) and photoinitiator in PBS, then polymerized under UV lamp to form a 20 weight percent PEG hydrogel (A). The gels were exposed to RAW 264.7 macrophages either through transwell inserts or direct seeding (B). Histology was performed on in vivo mouse subcutaneous implants after 7 days (C) and the inflammatory cell layer thickness was analyzed relative to non-therapeutic PEG hydrogel (D). Scale bar=50 μm, star indicates location of gel, arrows indicate inflammatory cell layer. Data presented as means with standard deviations as error bars. A one-way ANOVA was performed with Tukey's post-hoc analysis. One symbol: p<0.05, two symbols: p<0.01, three symbols: p<0.001.

FIG. 16 is a drawing showing potential applications/situations that can be resolved using the teachings of the present invention.

FIG. 17 is a drawing showing the toll-like receptor knock-outs.

FIG. 18 is a drawing showing the role of toll-like receptors in FBR.

FIG. 19 is a drawing depicting strategies to test the knockout of TLR 2 and 4 in vivo.

FIG. 20 is a series of images depicting the FBR in wt and knockout models.

FIG. 21 is a graph depicting the results quantifying the FBR in vivo.

FIG. 22 is a set of graphs depicting the results quantifying the FBR in vivo.

FIG. 23 is a set of graphs depicting the results quantifying the FBR in vivo.

FIG. 24 is a drawing with six graphs depicting pro-inflammatory cytokine expression

### DETAILED DESCRIPTION OF THE PREFERRED EMBODIMENT

Each year over 1 million Americans receive a life changing total knee or hip replacement, and the market is expected to increase due to the increasing prevalence of musculoskeletal disorders and disease. While orthopedic implants improve the quality of life for millions of patients, they are susceptible to failure. One common mode of implant failure that plagues virtually all synthetic orthopedic implants, is poor osseointegration due to fibrous tissue formation at the implant surface, which can lead to implant loosening. This type of failure occurs in 5-10% of patients with joint arthroplasty and requires revision surgery, which is costly and has a high complication rate. This fibrous tissue originates from the foreign body response (FBR), which is a ubiquitous response mediated by the innate immune system. It begins with inflammation and culminates in the production of a dense avascular fibrotic capsule that acts as a barrier to osseointegration, predisposing implants to failure. Increased fibrous tissue is found surrounding failed orthopedic implants suggesting an exacerbated FBR may be responsible for poor osseointegration and aseptic implant failure.

Recent evidence shows that macrophages are the innate immune cell that orchestrates fibrous encapsulation of implants. Through our research into this process, we discovered two cell surface receptors (TLR2 and TLR4) that are responsible for mediating the FBR. Key to our discovery was that TLR2 and TLR4 differentially contribute to the material-dependent FBR, and that blocking both could inhibit the FBR across a wide range of biomaterials. We also discovered that macrophages in the FBR become resistant to apoptosis leading to their persistence. This resistance occurs through the up-regulation of the intracellular protein, cFLIP. By knocking down cFLIP in macrophages, the FBR was partially attenuated. Together, these recent discoveries have identified three novel therapeutic targets that should prevent the FBR and improve osseointegration around orthopedic implants.

Using knowledge of these three novel targets, materials can be developed that limit the foreign body response to implants and reduce the occurrence of revision surgeries. We have identified compounds that inhibit both the cFLIP and TLR targets and have a linking mechanism developed that will bind these compounds to known biomaterials, thereby releasing the inhibitors in the presence of macrophages. We have applied this strategy to a hydrogel with a small molecule inhibitor of TLR2 and demonstrated partial attenuation of the FBR in a mouse model. Broader attenuation will be achieved using small molecule inhibitors of TLR2 and TLR4, with or without an inhibitor of cFLIP.

According to aspects of the invention, implantable biomaterials can be modified by attaching small molecule inhibitors (of TLR and cFLIP signaling) to the surface via a degradable linker. This linker will be sensitive to macrophage elastase, an enzyme produced primarily by macrophages. As such, the small molecules are released only when macrophages are present. This strategy has been applied to a hydrogel with small molecule inhibitor of TLR2 (T6167923) and attenuation of the FBR in a mouse model was demonstrated. Various strategies can be employed to immobilize small molecules that inhibit both TLR2 and TLR4, with and without cFLIP inhibitors, to releasably affix the inhibitor to orthopedic-relevant biomaterials (e.g., polyetheretherketone, silicone) using an oxygen plasma/silanization method. The concentration of the inhibitors, their combination, and linker degradation rate can be varied to evaluate the FBR using an established in vitro model and in vivo mouse model.

The foreign body response (FBR) is a formidable reaction that occurs to any non-biological implantable biomaterial and results in fibrous encapsulation. Non-specific protein adsorption is the first stage of the FBR and is thought to initiate the response by activation of innate immune cells. Here we show that Toll-like receptors (TLRs) 2 and 4 are the primary receptors responsible for recognizing surface adsorbed proteins as damage associated molecular patterns (DAMPs) and they determine the material dependent FBR. An in vitro model using multiple biomaterials identified that macrophages, not neutrophils, respond to surface-adsorbed plasma via TLR2 and/or TLR4 and that deletion of both were required to inhibit activation across all materials. In the more complex in vivo environment, simultaneous deletion of TLR2 and TLR4 nearly abrogated the FBR to multiple biomaterials and eliminated the material dependencies in vivo in a subcutaneous mouse model. Deletion of either TLR2 or TLR4 showed either no effect or a partial reduction, depending on the material, demonstrating that TLRs determine the material-dependent FBR in vivo. Collectively, we identified TLR2 and TLR4 as necessary receptors for the FBR and implicate macrophage recognition of DAMPs of surface-adsorbed proteins, which vary depending on the material, as the main driver initiating the FBR. Our findings establish TLR2 and TLR4 as therapeutic targets to evade the FBR across a range of implantable materials.

When a biomaterial is implanted into the body, endogenous proteins rapidly adsorb to its surface. These surface-adsorbed proteins may originate from different sources including blood plasma, tissue injury-associated cell death, and degraded extracellular matrix. The surface chemistry of the biomaterial determines the type, amount, and confirmation of the adsorbed proteins. Immune cells interact with the biomaterial implant through surface-adsorbed proteins, which is thought to initiate a foreign body response (FBR). The FBR is an innate immune response characterized by chronic inflammation and fibrotic encapsulation of the biomaterial by an avascular collagenous capsule. The FBR can negatively impact the function of biomaterial implants and lead to failure, such as aseptic loosening of orthopedic implants, contraction of cosmetic implants causing rupture, and prevention of tissue integration and regeneration in tissue engineering scaffolds. While the biomaterial chemistry and properties influence the severity of the FBR, essentially all non-biological biomaterials elicit an FBR. However, the mechanisms that cause the FBR are still poorly understood.

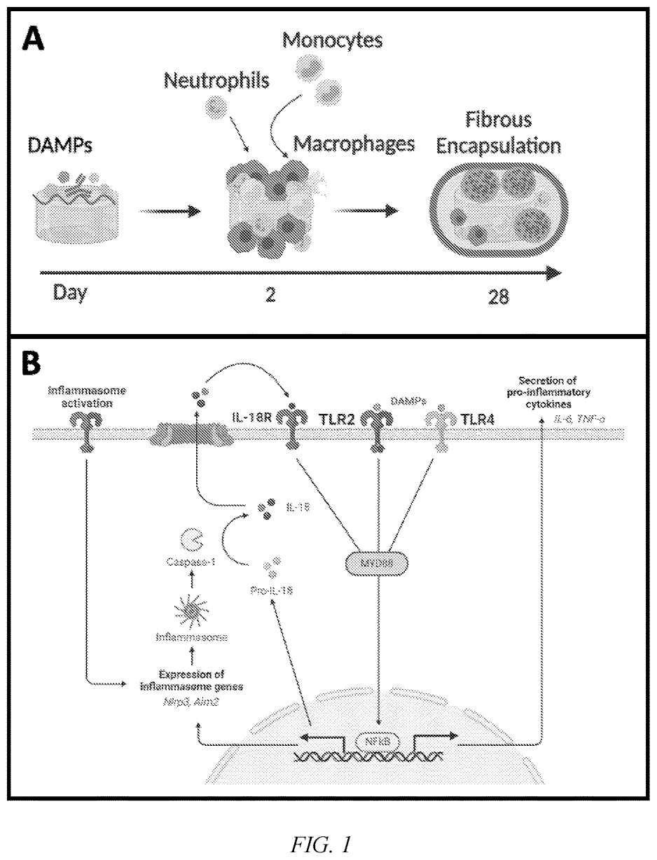

Surface-adsorbed proteins are thought to be recognized by neutrophils and macrophages during the initial stages of the FBR (FIG. 1A). Once adsorbed, these proteins may act as damage associated molecular patterns (DAMPs) through unfolding of otherwise non-inflammatory proteins or directly from proteins or other molecules released or degraded during tissue injury. Toll-like receptors (TLRs) are pattern recognition receptors that are capable of recognizing DAMPs to elicit a sterile inflammatory response. Of the TLRs, TLR2 and TLR4 possess the ability to bind DAMPs and activate nuclear factor kappa B (NF-κB) to induce transcription of pro-inflammatory cytokines (FIG. 1B). Proteins adsorbed to the surface of biomaterials in vitro activate NF-κB through TLR signaling, and specifically TLR2. TLR4 partially mediates the FBR to silicone implants in vivo, but deleting TLR4 did not affect the FBR to poly(ethylene terephthalate), indicating that TLR4-mediated signaling in the FBR is dependent on the biomaterial. There are many types of DAMPs that exist, some of which may be recognized by a single TLR (i.e., TLR2 or TLR4) while others may be recognized by multiple TLRs (TLR2 and TLR4), which creates redundancy in their signaling. This raises the question as to whether TLR2 and TLR4 via their recognition of multiple DAMPs are both involved in the FBR and whether they act in a redundant manner to induce inflammation.

Herein, this study tests the hypothesis that both TLR2 and TLR4 mediate the FBR in a material dependent manner via surface-adsorbed proteins, and that simultaneous deletion of both TLR2 and TLR4 is required to reduce the material-dependent inflammation and the subsequent FBR. To test this hypothesis, first we investigated the role of TLR2 and TLR4 using a simplified in vitro model of innate immune cells cultured on different biomaterials with surface-adsorbed plasma. Plasma was chosen to mimic the main surface-adsorbed proteins found in vivo. This study assessed neutrophils, which are considered the first responders in the FBR, and macrophages, which have been identified as key drivers of the FBR. Since macrophages and neutrophils have TLRs, both immune cells could contribute to initiating the FBR through their recognition of surface-adsorbed proteins via TLRs. Macrophages and neutrophils were isolated from TLR2^{-/-}, TLR4^{-/-}, and double knockout (TLR2-/-TLR4-/-or DKO) mice and compared to those isolated from wildtype (Wt) mice. Second, we investigated the role of TLR2 and TLR4 in the material-dependent FBR in vivo by evaluating the inflammatory response and the fibrous capsule generated using a mouse subcutaneous implant model. The in vivo environment is more complex and includes surface-adsorbed proteins derived not only from blood proteins found in tissue fluid, but also from intracellular proteins and the extracellular matrix that result from tissue injury. Moreover, in vivo studies are necessary to assess fibrous encapsulation, the final stage of the FBR. The FBR was assessed in Wt mice and compared to single knockouts for TLR2 (TLR2^{-/-}) and TLR4 (TLR4^{-/-}) and in the double knockout (TLR2^{-/-}TLR4^{-/-} or DKO).

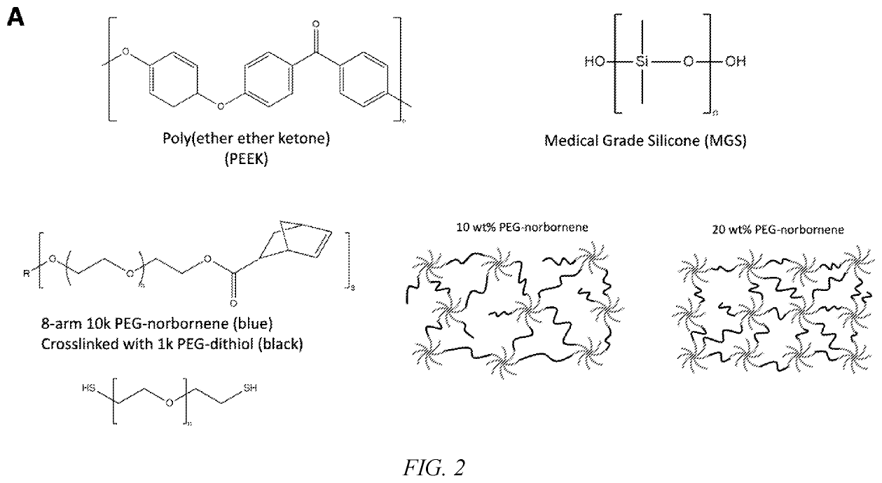

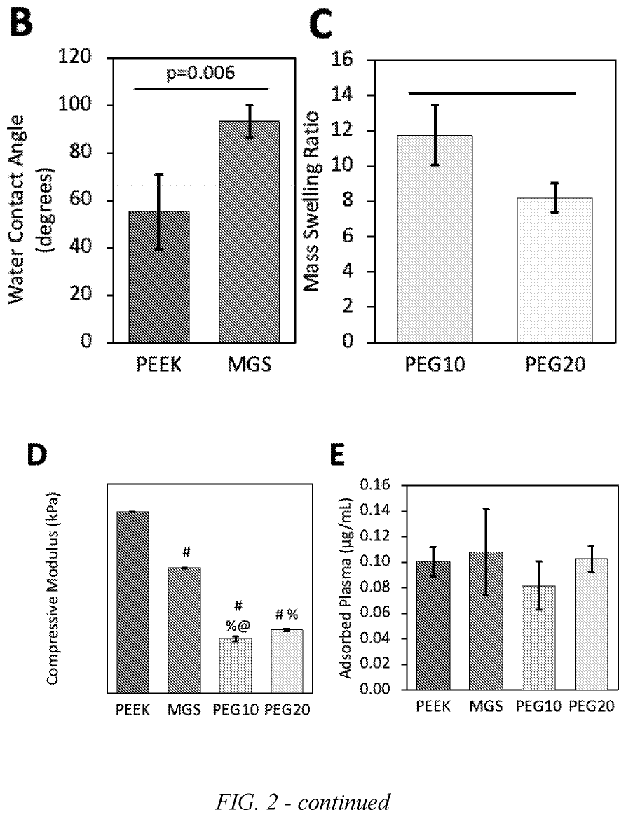

Four different biomaterials were selected for this study (FIG. 2A). Two of the biomaterials are currently used in FDA-approved implantable medical devices, poly (ether ether ketone) (PEEK) and medical grade silicone (MGS). PEEK has gained increasing interest in implantable medical devices for dental, orthopedic, and cardiovascular implants due to its high modulus and radiolucent properties. MGS is widely used in many implants including finger joint prostheses, breast augmentation, and trachea reconstruction. Both PEEK and MGS cause fibrous encapsulation. Poly(ethylene glycol) (PEG) hydrogels are one of the most widely used hydrogels for applications in tissue engineering, drug delivery, and coatings for medical devices. PEG hydrogels were formed by a thiol-norbornene click reaction, which is of interest for its high specificity and efficient reaction that enables orthogonal crosslinking and its spatiotemporal control offered through a step-growth photopolymerization reaction. A relatively uniform network structure is formed through this polymerization reaction which by altering its formulation via monomer concentration leads to different crosslink densities and hence different hydrogel stiffness (FIG. 2A). This approach allowed us to investigate the effect of hydrogel stiffness on the FBR without altering the chemistry of the material. Overall, this study determined that both TLR2 and TLR4 are the primary receptors involved in the FBR to multiple biomaterials, and together TLR2 and TLR4 determine the material-dependent FBR. Moreover our in vitro findings suggest that macrophages, not neutrophils, are the main responders to surface-adsorbed plasma via TLRs. This study provides direct evidence for DAMPs via TLR signaling as a major signaling pathway in the FBR.

### Example 1—Material Properties do not Impact the Extent of Protein Adsorption

As material properties could impact the total amount of proteins that adsorb to a biomaterial and in turn influence immune cell response, we first characterized each biomaterial for its hydrophobicity or water swelling capabilities. The hydrophobicity of MGS and PEEK was measured by water contact angle (FIG. 2B) and found to be 93° for MGS, which was higher (p=0.006) than PEEK at 55°. A water contact angle below 65° shown by the dotted line in FIG. 2B is considered hydrophilic. Thus, we conclude that MGS is hydrophobic, while PEEK is hydrophilic. Because hydrogels are inherently hydrophilic and absorb water, the extent of water content was assessed through mass swelling ratio, defined as the swollen mass relative to the dry polymer mass. The mass swelling ratio increased (p=0.006) from a value of 8 for PEG20 hydrogels to 11 for PEG10 hydrogels (FIG. 2C), indicating that the latter absorbed three times more of its dry mass in water than the former. The mechanical properties are another factor that could impact the severity of the FBR. The compressive modulus for PEEK was the highest (p≤0.05) at 1.16 GPa, followed by MGS at 15.4 MPa (FIG. 2D). The modulus was higher (p=0.01) for PEG20 at 130 kPa than PEG10 at 67 kPa. Plasma proteins are by far the most abundant proteins found on biomaterial implants. Thus, plasma was used to assess the extent to which biomaterial hydrophilicity and swelling impacted the extent of protein adsorption. To better mimic the in vivo environment whereby strongly and loosely adsorbed proteins are present, plasma was applied to each biomaterial and removed without rinsing the material. Material type was not a significant factor in the total amount of surface-adsorbed plasma (FIG. 2E). Overall, these results demonstrate that despite the differences in chemistry and material properties, the total amount of protein adsorption from plasma was independent of material properties. However, the type and nature of how these proteins interact with each biomaterial could be different.

### Example 2—Neutrophil Activation is Material Dependent and does not Require TLR2 and TLR4

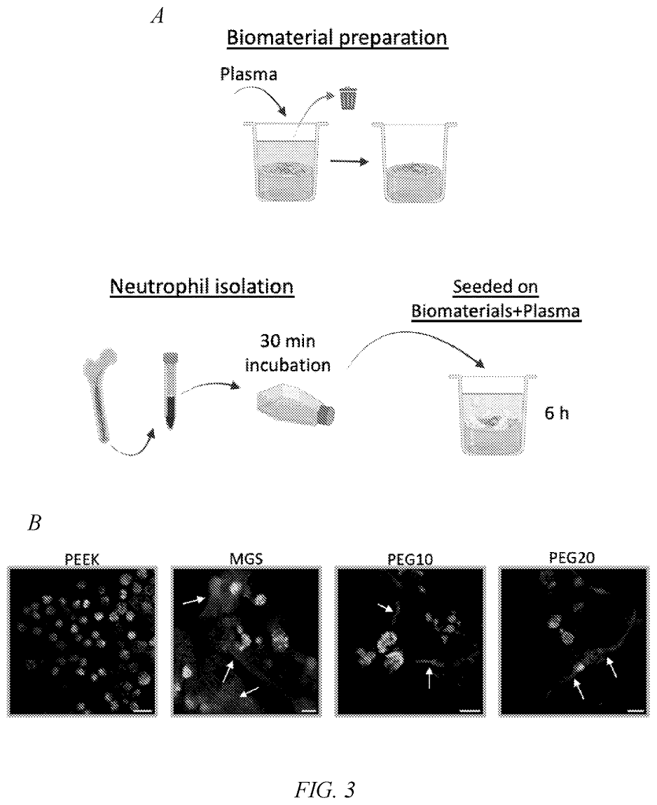

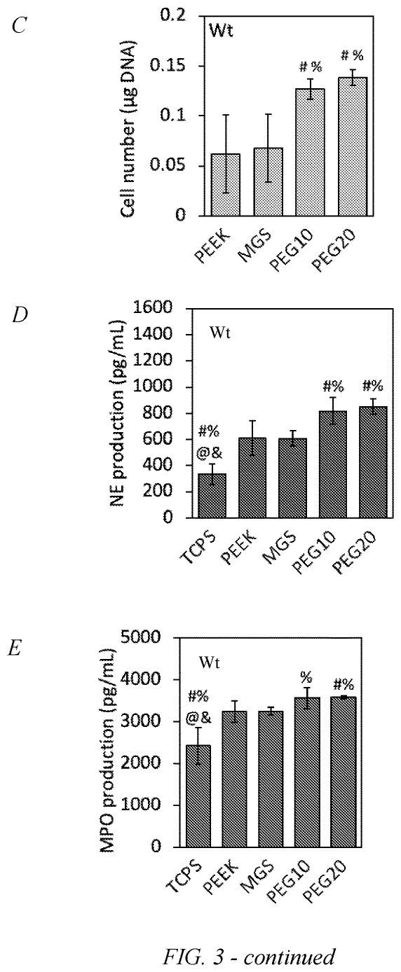

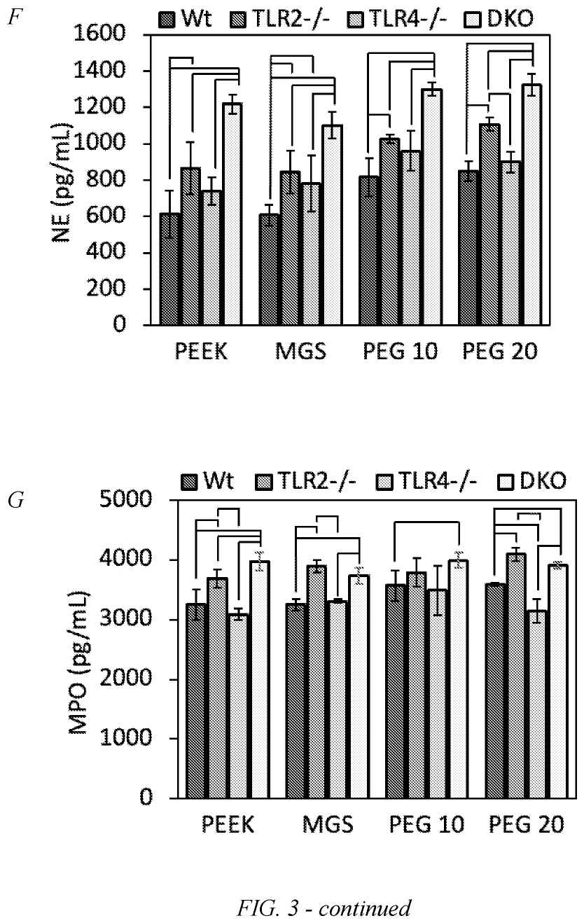

Neutrophils are considered the first responders in the FBR. To determine if neutrophils mediate the FBR via surface adsorbed molecules using TLRs, mouse neutrophils were isolated, and the neutrophil response to each biomaterial was assessed. To simulate the in vivo environment, plasma was pre-adsorbed to each biomaterial, and then neutrophils were seeded on top of the material and cultured in chemically defined (serum-free) medium (FIG. 3A). In this in vitro model, neutrophils interact with the biomaterial via surface-adsorbed plasma. Neutrophils do not readily attach to surfaces in vitro, thus the surface-absorbed plasma both encourages neutrophil attachment and simulates protein adsorption in vivo. Neutrophils attached to the surface of all biomaterials, which was confirmed by positive staining for nuclei and F-actin (FIG. 3B), and appeared intact and round with their lobed nuclei clearly visible, which is consistent with other reports. On MGS, there were extracellular regions that stained positive for DAPI, which may represent neutrophil extracellular trap (NET) formation similar to that shown in other biomaterial studies. The PEG10 and PEG20 materials also exhibited some evidence of a few nuclei trails, but not to the extent observed on MGS. On PEEK, all the cells retained their normal lobed nuclei with no evidence of nuclei trails or NET formation. Studies have reported NET formation on substrates with increased material stiffness and decreased hydrophilicity. The total number of cells was estimated by DNA content at 6 h post-seeding. The PEG10 and PEG20 hydrogels had a higher (p=0.03) number of neutrophils when compared to PEEK and MGS (FIG. 3C). It should be noted that the cell number was quantified after removing the media (no washing) to minimize losing lightly adhered cells; on the contrary, the staining requires multiple wash steps. Thus, the apparent discrepancy between the images and cell number is attributed to the loss of lightly adhered cells during the wash steps for imaging.

Neutrophil activation was assessed as a function of biomaterial type and measured by neutrophil elastase (NE) and myeloperoxidase (MPO) production (FIG. 3D,E). Tissue culture polystyrene (TCPS) without pre-adsorbed plasma was used as a control substrate. NE (0.3 ng ml-1) and MPO (2.3 ng ml-1) were detected in the culture medium of neutrophils on TCPS and at concentrations that are consistent with prior reports for naïve neutrophils after accounting for cell number. When neutrophils were cultured on the biomaterials with surface-adsorbed plasma, there was an increase (p<. 05) in NE and MPO that was biomaterial dependent (FIG. 3D). Other studies have reported an increase in NE and MPO on biomaterial surfaces when compared to the TCPS. In addition, studies have found that neutrophil activation is decreased on softer and more hydrophilic surfaces. Nonetheless, these findings demonstrate that biomaterials, possibly via the surface-adsorbed plasma, are able to activate neutrophils in vitro.

To determine whether TLR2 and/or TLR4 were required for biomaterial-induced activation of neutrophils, neutrophils isolated from the single KO and DKO mice were seeded onto each biomaterial with surface-adsorbed plasma, and their response was compared to Wt neutrophils. Deleting either TLR2 or TLR4 or both in neutrophils did not decrease production of NE (FIG. 3F) or MPO (FIG. 3G) across all biomaterials. The exception is PEG20 whereby TLR4 partially contributed to neutrophil activation, but deleting both did not decrease MPO production. These results indicate that biomaterial-induced activation via surface adsorbed plasma, for the most part, does not require TLR2 and/or TLR4.

Collectively, these findings indicate that neutrophils attached to and were activated by each biomaterial with surface-adsorbed plasma. Neutrophils largely retained their lobed nuclei on the biomaterials with some possible evidence of NET formation on MGS. It is concluded that the biomaterial-dependent neutrophil activation does not require TLR2 or TLR4 on neutrophils.

### Example 3—Surface Adsorbed Plasma Activates Macrophages in a Biomaterial-Dependent Manner

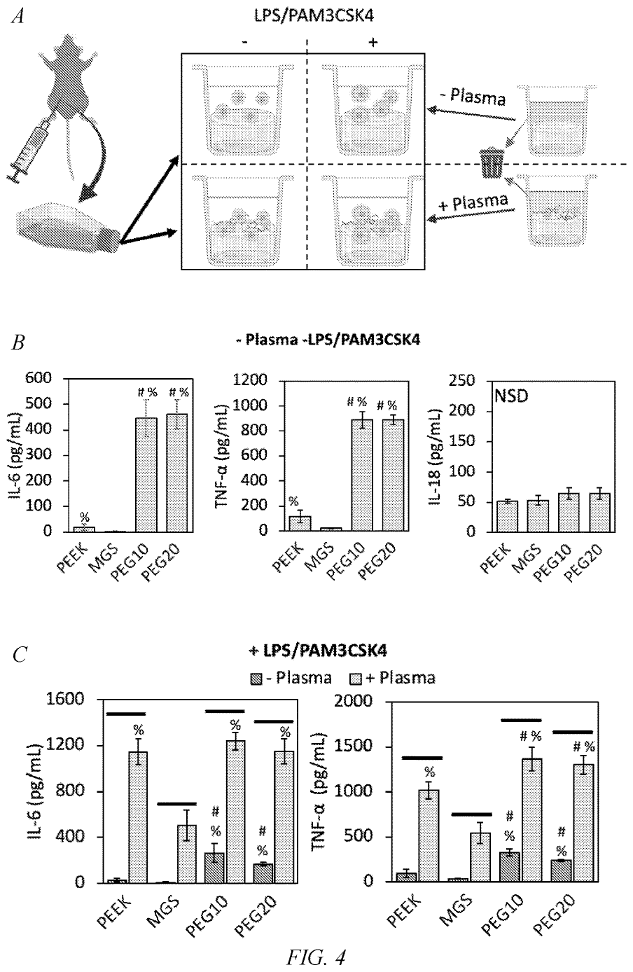

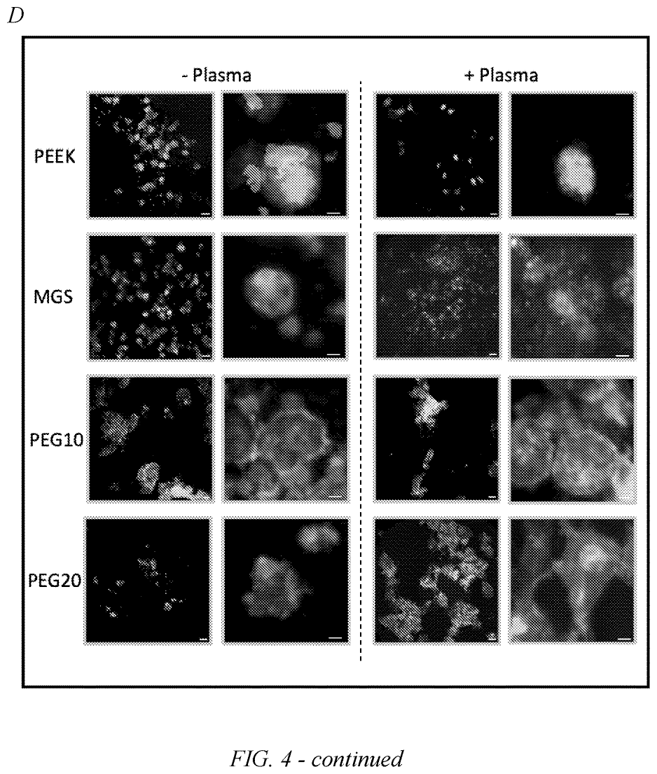

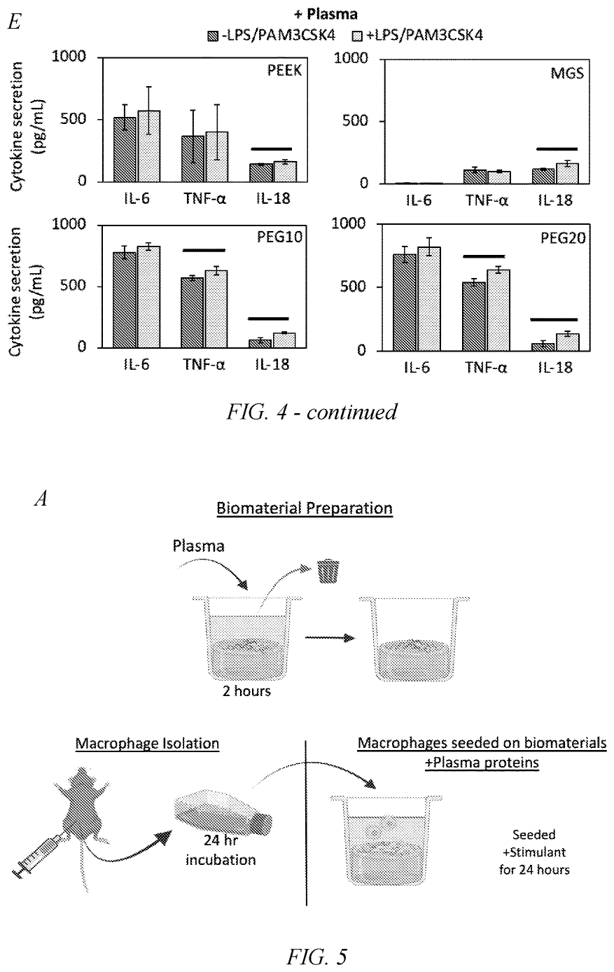

Experiments were conducted to determine if macrophages could sense biomaterial surface adsorbed molecules, the first step in the FBR, using TLRs. An in vitro model was established to mimic aspects of the in vivo environment. Plasma was pre-adsorbed to each biomaterial, and then macrophages were seeded on top of the material and cultured in chemically defined (serum-free) medium (FIG. 4A). In this in vitro model, macrophages interact with the biomaterial via surface adsorbed plasma similar to that in vivo during the FBR. Pre-activation of macrophages in vitro is necessary to mimic elements of the FBR observed in vivo, which can be attributed to the fact that pro-inflammatory macrophages respond differently to surface-adsorbed proteins than naïve macrophages. Peritoneal macrophages express substantially more Arg1 than Tnfa (FIG. 8), suggesting that these naïve macrophages mimic a M2-like pro-healing state rather than a M1-like pro-inflammatory state. Thus, the macrophages were treated with a low concentration (1 ng ml⁻¹) of soluble pro-inflammatory stimulant (0.5 ng ml⁻¹ LPS+0.5 ng ml-1 PAM3CSK4), concentrations that are well below those normally used to activate macrophages. This low dose priming was used to allow one to determine the contribution of the biomaterial and surface-adsorbed proteins. To understand the impact of the different components in the in vitro model, a series of experiments were performed to assess the impact of the biomaterial itself, the surface-adsorbed plasma, and the low-level priming treatment.

Macrophage response to each biomaterial was first assessed without surface-adsorbed plasma and without LPS+PAM3CSK4 treatment to determine if the biomaterial itself could induce macrophage activation (FIG. 4B). Macrophage attachment was confirmed by visual inspection under a bright field microscope for all biomaterials except for PEEK, which is opaque. Macrophages seeded on PEEK and MGS produced low levels of the pro-inflammatory cytokines interleukin-6 (IL-6) and tumor necrosis factor α (TNF-α) with PEEK inducing 9-fold higher (p≤0.014) IL-6 and 5-fold higher (p≤0.011) TNF-α levels than that induced by MGS. On the contrary, PEG10 and PEG20 induced up to 237-fold higher (p≤0.014) IL-6 and TNF-α compared to both PEEK and MGS. IL-18 is constitutively expressed by macrophages and produced in pro-protein form, that requires inflammasome activation to produce the active form. Macrophages produced low levels of active IL-18, but there was no material dependence, suggesting a basal level of IL-18 production, which is consistent with prior reports.

Next, the impact of surface-adsorbed plasma on activation of macrophages that are primed towards a pro-inflammatory stimulant with LPS+PAM3CSK4 was assessed. IL-6 and TNF-α production by macrophages cultured on biomaterials without and with surface-adsorbed plasma was compared (FIG. 4C). When macrophages interacted with the biomaterials via surface-adsorbed plasma instead of the biomaterial directly, both IL-6 and TNF-α production were significantly higher across all biomaterials, but the extent was material-dependent. For PEEK and MGS, IL-6 was 42-77-fold higher and TNF-α was 11-16-fold higher. For PEG10 and PEG20, IL-6 was 4.7-6.9-fold higher and TNF-α was 4-5.4-fold higher. The material dependence differed when comparing without and with surface-adsorbed plasma, notably for PEEK. Without surface-adsorbed plasma, PEEK was more similar to MGS and with surface-adsorbed plasma, PEEK was more similar to the PEG hydrogels. To determine whether the increased pro-inflammatory cytokine production was in part attributed to macrophage morphology due to the presence of surface-adsorbed plasma, macrophages seeded on each biomaterial were stained for F-actin and nuclei and visualized by confocal microscopy. For PEEK, macrophages exhibited a round morphology, which was visually similar with or without surface-adsorbed plasma. For MGS, macrophages exhibited a round morphology without surface-adsorbed plasma. With surface-adsorbed plasma, the F-actin staining was more faint, and the cells appeared more spread out. For PEG10, macrophages appeared in aggregates with no observable differences with or without surface-adsorbed plasma. For PEG20, macrophages appeared to be more spread out in the presence of pre-adsorbed plasma. Importantly, these results show that macrophages attached to the biomaterials in the absence or presence of plasma proteins. While there were some differences in morphology in the presence of surface-adsorbed plasma, there was no obvious correlation between cell morphology and the biomaterial-dependent pro-inflammatory cytokine production.

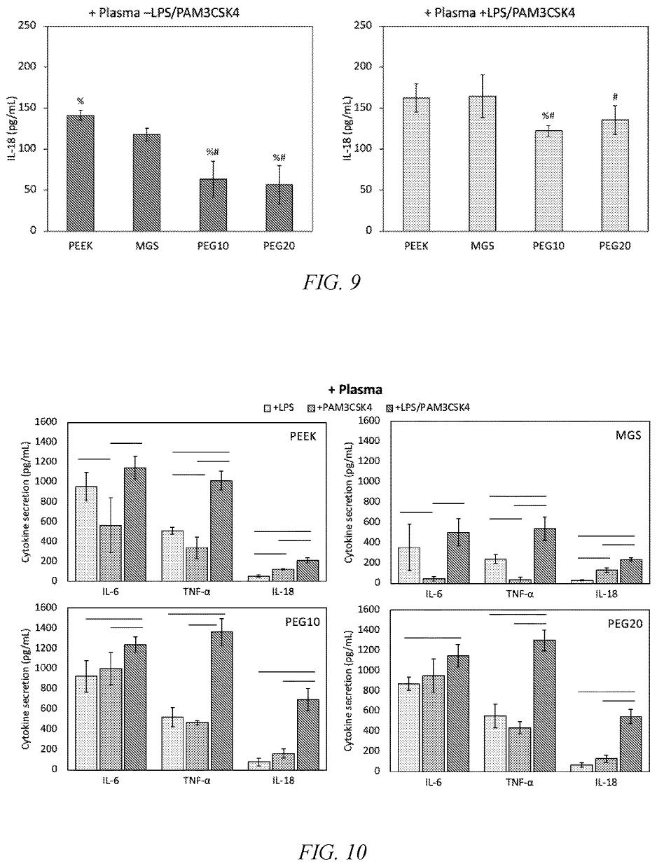

Finally, the impact of pre-treatment with low-levels of LPS+PAM3CSK4 on production of IL-6, TNF-α, and IL-18 by macrophages cultured on each biomaterial with surface adsorbed plasma was assessed (FIG. 4E). After 24 h, there were no differences in IL-6 production across all biomaterials when stimulated with low dose LPS+PAM3CSK4. TNF-α production was very slightly, but still significantly, higher in the presence of the PEG hydrogels with LPS+PAM3CSK4. Pre-treatment with LPS+PAM3CSK4 led to a moderate but significant increase in IL-18 production across all biomaterials (15% p=0.027 for PEEK; 41% p=0.005 for MGS; 93% p=0.0008, for PEG10; 147% p=0.00008 for PEG20). When comparing IL-18 with LPS+PAM3CSK4 treatment across the materials, there was a material dependence (FIG. 9), indicating that surface-adsorbed plasma induces IL-18 production but in a material-dependent manner. Taken together, these findings show that the low levels of LPS+PAM3CSK4 and surface-adsorbed proteins both enhance IL-18 production in a biomaterial-dependent manner. In agreement with prior studies, macrophages primed to be in a pro-inflammatory state respond to surface-adsorbed proteins in a biomaterial dependent manner.

Collectively, these findings indicate that while the biomaterial itself is capable of activating macrophages, the presence of surface-adsorbed plasma leads to increased pro-inflammatory cytokine production in macrophages in a biomaterial-dependent manner. The plasma-induced response is exacerbated when macrophages are primed towards a pro-inflammatory state (i.e., with LPS and PAM3SCK4 treatment).

### Example 4—Both TLR2 and TLR4 are Required for Macrophage Activation on Biomaterials with Surface-Adsorbed Plasma

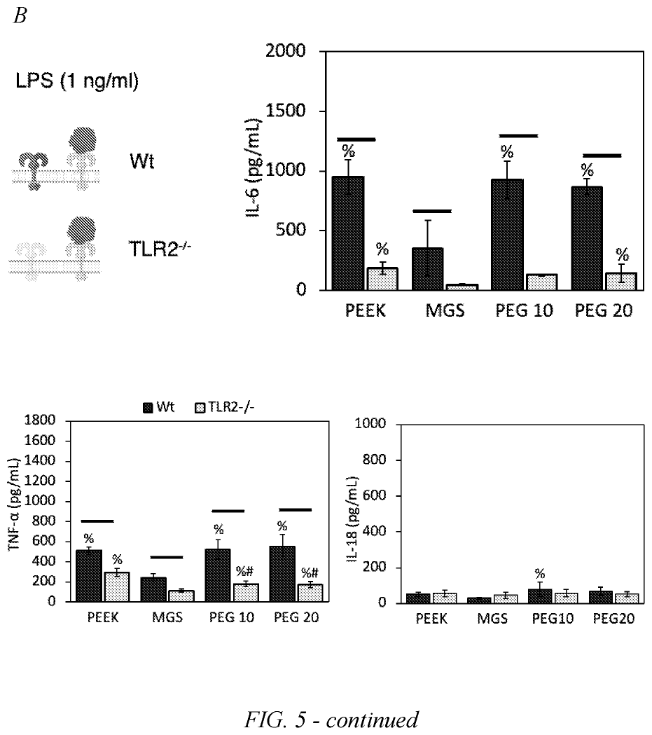

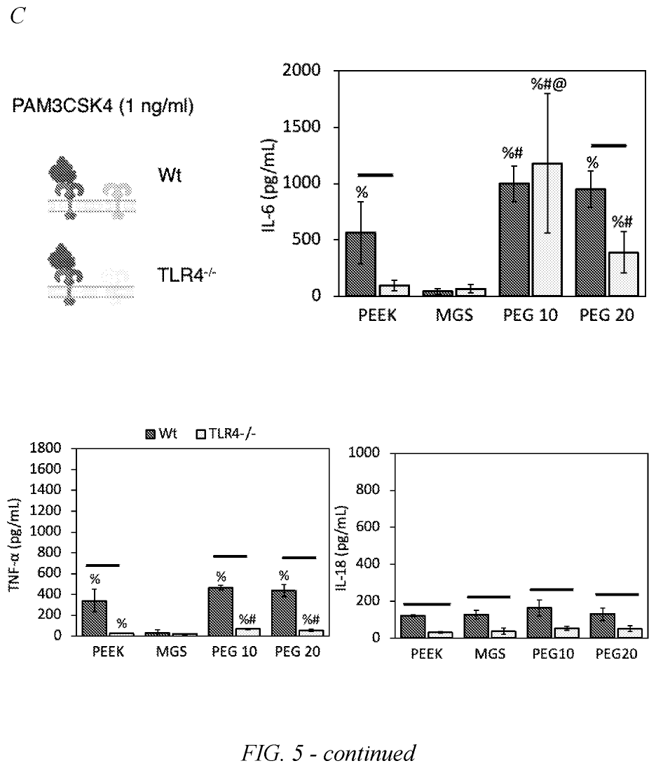

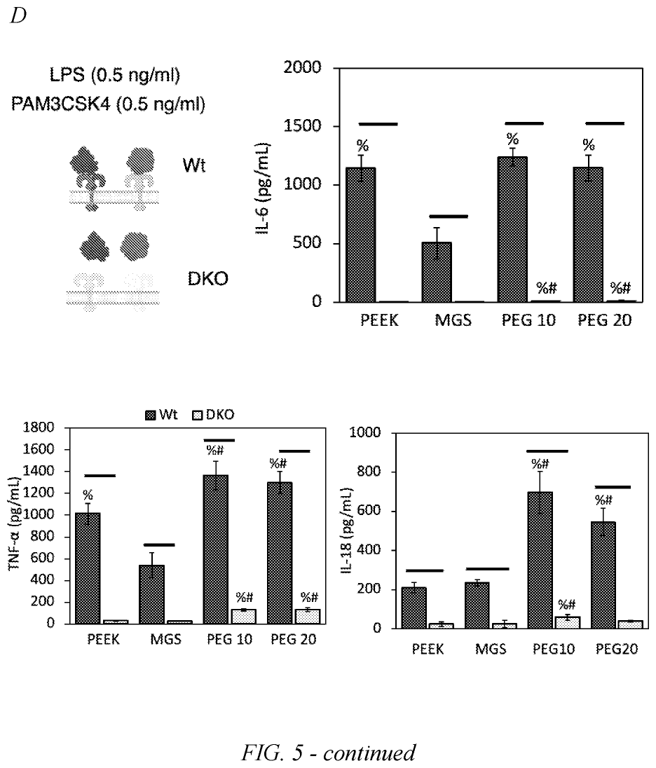

Using the in vitro model established above, TLRs were investigated for their ability to mediate the plasma-induced biomaterial activation of macrophages. Macrophages were isolated from Wt, single KO, and DKO mouse strains and seeded on biomaterials that had been pre-treated with plasma (FIG. 5A). Macrophages were primed with 1 ng ml-1 of the pro-inflammatory stimulant. For the studies with TLR2^{-/-} macrophages, LPS (1 ng ml-1) was used because TLR2 ablation will not affect the response to LPS, changes in cytokine production reflect the effects of the biomaterial and adsorbed plasma. Likewise, macrophages were pre-treated with PAM3CSK4 (1 ng ml-1) for the studies with TLR4^{-/-} macrophages; in this case, any effects of TLR4 deletion will reflect the effects of the biomaterial and adsorbed plasma. For the studies investigating DKO macrophages, macrophages were primed with both LPS and PAM3CSK4 (0.5 ng ml-1 each). Wt macrophages in each experiment were treated with the same priming stimulants. One limitation of the DKO macrophage studies is that ablation of TLR2 and TLR4 simultaneously will affect the response to both the priming agents and the biomaterial plus plasma, making it difficult to conclusively ascribe the effects of the DKO, although the extremely low doses of priming agents used likely minimize this concern. The effects of the biomaterials via surface-adsorbed proteins and the priming agents were most pronounced with LPS and PAM3CSK4 (comparing Wt response to LPS alone or PAM3CSK4 alone; FIG. 10) for each biomaterial.

To assess the role of TLR2 in macrophage activation as a function of biomaterial type, TLR2^{-/-} macrophage response was compared to Wt cells (FIG. 5B). Deleting TLR2 led to a ≥43% decrease (p≤0.032) in IL-6 and TNF-α production in the presence of all materials. IL-18 levels were relatively low ranging from 29 to 80 μg ml-1 in the Wt strain with PEG10 producing the highest level and MGS producing the lowest level. TLR2 deletion had no significant effect on IL-18 production. These results indicate that across all materials there are surface-adsorbed plasma molecules that solely activate TLR2; although, the extent is material dependent, and also varies by cytokine readout. These results also show that TLR2 is not involved in the basal levels of IL-18 production (i.e., for the biomaterial only, comparing to FIG. 4B).

To assess the role of TLR4 in macrophage activation to biomaterials with surface-adsorbed plasma, the TLR4^{-/-} macrophage response was compared to Wt cells (FIG. 5C). Deleting TLR4 attenuated macrophage activation in a biomaterial dependent manner. Both IL-6 and TNF-α production were reduced (p≤0.016) in the TLR4^{-/-} macrophages in the presence of PEEK or PEG20. IL-6 and TNF-α production by macrophages on MGS was low (33-44 μg ml⁻¹) for the Wt, and deleting TLR4 had no effect. On PEG10, there was an 86% reduction (p<0.001) in TNF-α production when TLR4 was deleted, but no significant effect on IL-6 production was observed. These results indicate that there are surface-adsorbed plasma molecules on the biomaterials with the exception of MGS that solely signal using TLR4. For PEG10, the role of TLR4 signaling differs for IL-6 (no effect) and TNF-α (has an effect). IL-18 production ranged from 124 to 165 μg ml⁻¹ in the Wt strain. IL-18 production was significantly decreased (p≤0.010) across all materials when TLR4 was deleted. This result indicates that surface-adsorbed plasma molecules solely signaling to TLR4 are responsible for the combined effect of surface-adsorbed proteins in macrophages primed with PAM3CSK4.

To assess the role of both TLR2 and TLR4 on macrophage activation to biomaterials with surface-adsorbed plasma, the DKO macrophage response was compared to Wt cells (FIG. 5D). Deleting both TLRs resulted in a >78% reduction (p≤0.001) in IL-6, TNF-α, and IL-18 production across all materials. Comparing the response of the Wt and DKO macrophages across the biomaterials shows that the material dependence observed in Wt macrophages is nearly, although not completely, abrogated in the DKO macrophages. In the DKO macrophages PEG10 and PEG20 induced greater (p≤0.005) IL-6 and TNF-α production compared to PEEK and MGS, while only PEG10 induced greater (p≤0.042) IL-18 production compared to PEEK and MGS. Thus, surface-adsorbed plasma molecules signaling through both TLR2 and/or TLR4 leads to pro-inflammatory cytokine production in a material-dependent manner.

Collectively, these findings demonstrate that macrophages primed to be in a pro-inflammatory state interact with surface-adsorbed proteins via TLR2 and TLR4 in a biomaterial dependent manner. These findings show that while surface-adsorbed proteins contribute the most significantly to macrophage activation (comparing FIGS. 3C and 3E), the nature by which the macrophages are primed impacts the extent of the response (FIG. 10). Nonetheless, these in vitro studies show that the macrophage response to surface-adsorbed proteins requires both TLR2 and TLR4 across all biomaterials tested.

### Example 5—the FBR is Material-Dependent

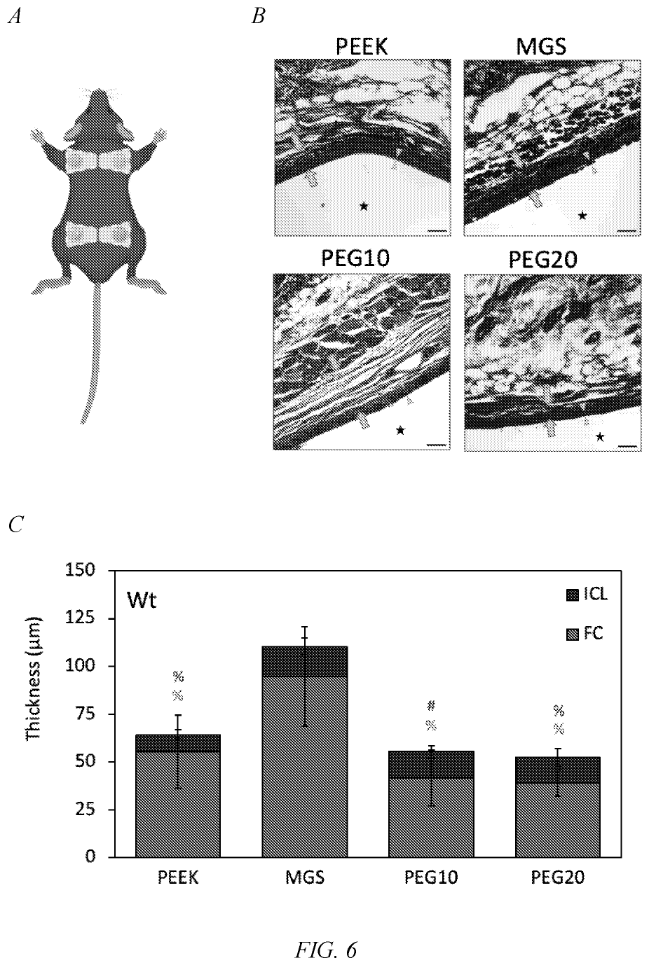

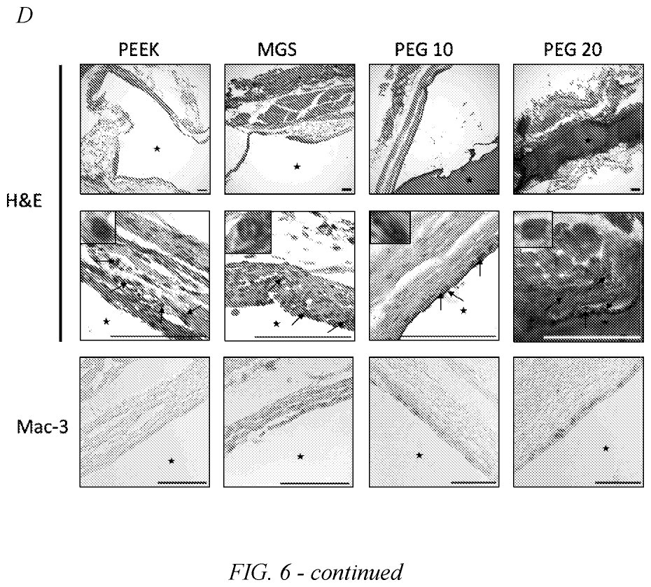

The in vitro findings shown herein implicate both TLR2 and TLR4 for their role in mediating the biomaterial-dependent macrophage response to biomaterials via the surface-adsorbed plasma. The effects were therefore investigated in the more complex in vivo environment. To quantify the FBR, the thickness of the inflammatory cell layer (ICL) adjacent to the implant was measured to assess the extent of inflammation. The hallmark of the FBR is the fibrous capsule (FC) and its thickness is an indicator of the severity of the FBR. Each biomaterial was implanted into a separate subcutaneous pocket of Wt mice (FIG. 6A) and the FBR analyzed by histology 28 days later. Representative microscopy images of Gömöri trichrome stained sections show an FBR induced by all biomaterials (FIG. 6B). A dense layer of cells can be seen at the implant surface across all materials. When quantified, the ICL was the thinnest for PEEK, appearing as a single layer of cells, and was significantly smaller than that induced by MGS or PEG10 (FIG. 6C). The FC thickness was greatest (p<. 05) for MGS compared to PEEK and both PEG hydrogels (FIG. 6C). There was no significant difference in the ICL or FC thickness between PEG20 and PEG10.

The presence of immune cells was also probed on day 28 to assess for chronic inflammation. Neutrophils around the implants were identified by their lobed nucleus in hematoxylin and eosin (H&E) stained sections (FIG. 6D). Neutrophils were found in the FC and the ICL. Macrophages around the implants were identified by positive staining for Mac-3 and were located primarily in the ICL (FIG. 6D). For PEEK, the Gömöri trichrome stained sections showed a single layer of inflammatory cells, which can be faintly identified in the sections stained with Mac-3 along the edge of the implant. Macrophages were clearly visible along the implant surface for MGS, PEG10, and PEG20. Visually, there were more macrophages along MGS when compared to the PEG hydrogels. No differences were observed between PEG10 and PEG20.

Taken together, these results show that the severity of the FBR is material dependent. Of the materials tested, MGS, which is the most hydrophobic material, caused the most severe FBR, with no observable differences arising from material stiffness (comparing PEG10 to PEG20). The presence of neutrophils and macrophages surrounding each biomaterial at day 28 indicate chronic inflammation, which is consistent with the presumed pathogenesis of the FBR. Collectively, these findings implicate material chemistry as a significant factor in the FBR.

### Example 6—TLR2 and TLR4 are Both Required for the FBR

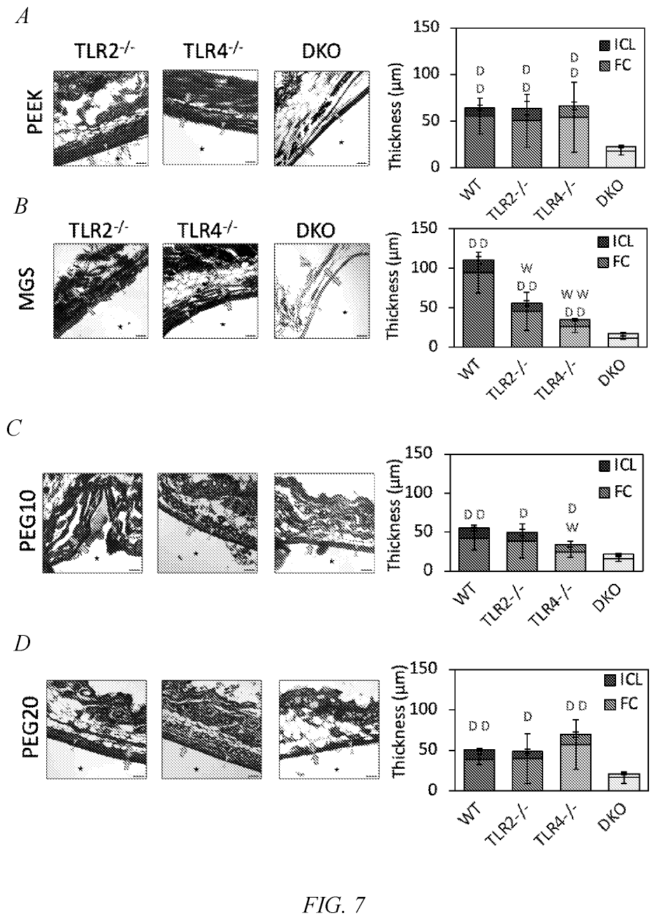

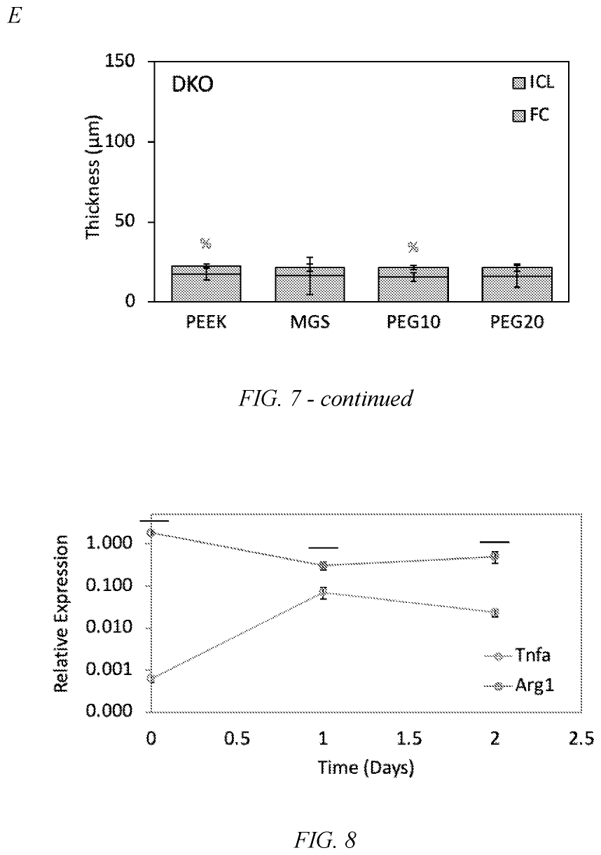

To assess the role of TLR2 and TLR4 in the FBR, each biomaterial was implanted into separate subcutaneous pockets for 28 days in TLR2^{-/-} and TLR4^{-/-} mice (single KOs) and the DKO mice and the FBR analyzed by histology (FIG. 7). Representative microscopy images of Gomori trichrome stained sections from the KOs along with the quantitative results, which include Wt (from FIG. 6B) are shown in FIG. 7A-D. With PEEK, there was no significant difference in the ICL or FC thickness in the single KOs compared to Wt mice (FIG. 7A). However, in the DKO, there was a 41% (p≤0.013) reduction in the ICL and 68% (p≤0.005) reduction in the FC compared to Wt mice. These results suggest that surface-adsorbed molecules act as DAMPs that can signal using both TLR2 and TLR4 in the FBR to PEEK, requiring deletion of both TLRs to observe a phenotypic change in the FBR. With MGS, the ICL was reduced (p=0.008) in the TLR4^{-/-} mice but was not affected significantly in the TLR2^{-/-} mice (FIG. 7B). The FC thickness was reduced (p≤0.013) in both single KOs compared to the Wt mice for MGS. Both ICL and FC thickness were reduced (p≤0.022) in the DKO mice when compared to the Wt and each of the single KOs for MGS. Contrary to PEEK, these results suggest that multiple DAMPs are generated on MGS, some that signal solely using TLR2, some that signal solely using TLR4, and some that signal via both TLRs. With PEG10, there was no significant difference in the ICL in the single KOs compared to the Wt mice, but the ICL was reduced (p=0.008) by 56% in the DKO when compared to Wt mice (FIG. 7C). The FC thickness was not significantly decreased in the in the TLR2^{-/-} mice but was reduced (p=0.031) by 42% in the TLR4^{-/-} mice for PEG10. The FC thickness was reduced (p=0.008) by 63% in the DKO when compared to the Wt mice for PEG10. With PEG20, there was no significant difference in the ICL or FC thickness in the single KOs compared to the Wt mice (FIG. 7D). However, in the DKO, the ICL was reduced (p=0.020) by 62% and the FC was reduced (p=0.008) by 58% compared to the Wt mice for PEG20. Thus, for the two PEG hydrogels, some surface-adsorbed molecules were sensed by TLR4 in the PEG10 but not in the PEG20 studies; however, the majority of the signaling occurs through both TLR2 and TLR4.

Comparing the FBR across all biomaterials in the DKO strain, PEEK, MGS, PEG10, and PEG20 showed a minimal ICL and FC thickness (17.6, 16.4, 15.6, and 16.2 μm, respectively) (FIG. 4E). There was no material dependence on the ICL. MGS had a slightly decreased (p=0.036, 0.022, respectively) FC thickness compared to PEG10 and PEEK, but there was no significant difference between PEG10, PEG20, and PEEK. These results demonstrate that TLR2 and TLR4 determine the material dependencies in the FBR and that their simultaneous deletion is required to mitigate the FBR across distinctly different biomaterials.

As show herein, TLR2 and TLR4 are required for the FBR that leads to fibrous encapsulation of multiple biomaterial implants. The material dependence in the FBR could largely be explained by differences in whether TLR2 or TLR4 or both are responding to the biomaterial. Importantly, these findings identified redundancy in TLR2 and TLR4 activation whereby inhibiting one or the other is not sufficient to fully prevent the FBR, but ablating both TLR2 and TLR4 led to near abrogation of the fibrous capsule induced by four different biomaterials. The in vitro findings provide evidence that DAMPs arising from surface-adsorbed plasma proteins activate macrophages, and this response occurs primarily through these same TLRs. The in vitro studies identified macrophages, not neutrophils, for their ability to be activated by surface-adsorbed plasma via both TLR2 and TLR4. Overall, this study provides evidence for the link between DAMPs, macrophages, and TLRs, specifically TLR2 and TLR4, that cause the material dependence of the FBR and showed that blocking both TLR2 and TLR4 could nearly abrogate fibrous encapsulation across four different biomaterials.

The material dependent FBR in vivo is driven by differences in DAMPs sensed by TLR2 and TLR4. TLR2 and TLR4 recognize DAMPs from surface-adsorbed proteins or other molecules that arise from tissue injury or from unfolded endogenous proteins. The in vitro studies confirmed that surface-adsorbed plasma can induce an inflammatory response in macrophages in a material-dependent manner. These findings confirm that plasma, which otherwise is not inflammatory, can produce DAMPs when adsorbed to a biomaterial surface. Deletion of TLR2 in vitro led to the partial reduction in pro-inflammatory cytokine production in the presence of MGS only, while deletion of TLR4 led to the partial reduction in cytokines induced by MGS and PEG10, but not PEEK or PEG20. These results indicate that there are surface-adsorbed DAMPs that signal exclusively to TLR4 or to TLR2, but that are present only on certain materials. On the contrary, the FBR to PEEK and PEG20 were not affected by deleting TLR2 or TLR4 individually. This finding suggests that essentially all surface adsorbed DAMPs on these materials are recognized by both TLRs. Of the four materials tested in this study, MGS is the most hydrophobic, which could lead to different protein interactions with the surface and explain the presence of TLR2-specific DAMPs, which was not found on the other materials. PEEK, PEG20, and PEG10 are hydrophilic but are distinctly different materials; PEEK is an ultra-stiff material that does not absorb water while both PEGs are relatively soft hydrogels containing >85% water. Despite these differences, PEEK and PEG20 exhibited similar FBRs in magnitude and TLR sensitivity while PEG10, which is the softest and most hydrophilic of all materials, showed a dependence on TLR4. In vitro the amount of adsorbed plasma proteins, which included strongly and loosely adsorbed proteins, was not statistically different between the biomaterials, implying that the way plasma proteins interact with a surface chemistry to produce DAMPs, such as by the types of proteins that adsorb, their conformation, and/or the ratio of strongly to loosely adsorbed molecules are dominating factors that determine the material dependent cell response. We purposely chose to examine the FBR to PEG10 and PEG20 because they have the same chemistry but different moduli. Interestingly the FBR was no different between these materials in the Wt or DKO mice suggesting that at least over this modulus range, stiffness did not modulate the FBR. However, the difference in the FBR in TLR4^{-/-} mice suggest that differences in the network structure and/or water content may have led to differences in how proteins interact with and hence DAMPs form on these hydrogels. Despite hundreds of proteins that adsorb to materials in vivo, our findings point to TLR2 and TLR4 as the primary pattern recognition receptors responsible for recognizing DAMPs on biomaterial implants. Our data furthermore demonstrate redundancy in TLR2 and TLR4 signaling, and thus inhibiting both is required to substantially dampen the FBR to biomaterial implants.

Macrophages were classically activated in a material-dependent manner by surface-adsorbed plasma via TLR2 and TLR4 signaling. Broadly, the in vitro studies provide direct evidence that plasma, when adsorbed to a biomaterial, produces DAMPs and induces an inflammatory response in macrophages that is mediated by both TLR2 and TLR4, consistent with the in vivo studies. While surface adsorbed proteins are believed to be the primary driver of macrophage activation in the context of the FBR, it is possible that the surface chemistry itself could be contributing. To this end, we also probed macrophage activation on the biomaterials in the absence of pre-adsorbed plasma. Interestingly, macrophages attached to all biomaterials without plasma proteins. It is possible that macrophages produce their own extracellular adhesion proteins that facilitate cell attachment. In the absence of surface adsorbed plasma, there was minimal macrophage activation on PEEK and MGS suggesting that surface-adsorbed plasma is the mechanism driving the inflammatory response to these biomaterials. Surprisingly, the PEG hydrogels themselves induced pro-inflammatory cytokine production. There was a stark difference in macrophage morphology on the PEG hydrogels compared to MGS and PEEK, where cells appeared aggregated on the surface of the PEG hydrogels. It is worth noting that the rinsing steps for staining and imaging remove loosely bound cells that were visually observed before staining. A possible explanation for the increase in inflammatory cytokines could be due to the reduced engagement of cell surface receptors with the biomaterial. Incorporating RGD into a PEG hydrogel attenuates macrophage activation in vitro and the FBR in vivo.

Taken together, our findings indicate that while macrophages can recognize and adhere to the underlying biomaterial, the presence of surface-adsorbed plasma causes significant activation. Interestingly the in vitro studies show that the hydrophilic materials (PEEK, PEG10, and PEG20) cause greater macrophage activation than the hydrophobic material MGS. An in vitro study showed that altering the hydrogel chemistry to increase its hydrophobicity reduced pro-inflammatory cytokine production by attached macrophages. The in vitro findings herein differ from the in vivo findings, which found MGS to induce a more severe FBR. The in vitro study focused on assessing the impact of surface-adsorbed plasma, and while plasma proteins represent the most abundant surface-adsorbed proteins in vivo, other sources of proteins associated with tissue injury are found on implants and have been shown to activate macrophages in vitro. Regardless of the exact source of DAMPs, the in vitro and in vivo findings determined that despite differences in the severity of the FBR in vivo or macrophage activation in vitro, the response is predominantly mediated through TLR2 and/or TLR4.

While neutrophils are present throughout the FBR, ablation of TLR2 and/or TLR4 in neutrophils did not affect biomaterial-induced neutrophil activation. In support of these findings, ablation of neutrophils using a neutrophil-depleting antibody did not impact fibrous encapsulation of alginate hydrogels. Neutrophil elastase was measured, which is required for the formation neutrophil extracellular traps (NETs) and myeloperoxidase which is involved in NET formation, as indicators of neutrophil activation. Several studies have shown that TLR signaling is not involved in NET formation. The present in vitro findings showed that neutrophil activation by biomaterials was independent of TLR2 and TLR4. These findings suggest that neutrophil signaling via TLRs is not a major contributor to the FBR, and it is surmised that the reduced FBR observed in the DKO mice was likely mediated by macrophages rather than neutrophils in response to surface-adsorbed DAMPs.

In conclusion, the findings from this study demonstrate that TLR2 and TLR4 are required for the FBR to different biomaterials and that their individual contribution depends on material chemistry in large part through the adsorbed proteins. Most interesting is that these findings implicate that the majority of the DAMPs that are formed by biomaterials signal through both TLR2 and TLR4 indicating a redundant signaling mechanism where blocking one of the receptors is insufficient to prevent the FBR. Overall, these results suggest that inhibition of both TLR2 and TLR4 simultaneously may be a viable therapeutic option to abrogate the FBR across a wide range of biomaterials.

### Example 7—Materials and Methods—Part 1

Biomaterials: Sterile MGS was purchased in 1.5 mm thick sheets from Invotec International. Cylindrical disks of MGS of dimensions 5 mm diameter were created using a sterile biopsy punch. PEEK was purchased in 1 mm thick sheets from Victrex, and 5 mm diameter cylindrical disks were created using a steel punch. The disks were sterilized by autoclaving. For the PEG thiol-norbornene (PEGNB) hydrogels, 8-arm PEG-norbornene (10,000 g/mol) was purchased from Biochempeg. All hydrogels were formed by photopolymerization. PEGNB hydrogels were formed at two different formulations, 20% (w/w) (PEG20) and 10% (w/w) (PEG10) and crosslinked with PEG-dithiol (1000 g/mol, Sigma) at a 1:1 thiol: ene ratio in PBS. Each monomer solution was combined with a photoinitiator (Irgacure 2959, BASF) at a final concentration of 0.05% (w/w). All solutions were sterile filtered and polymerized under 352 nm light at 6 mW/cm² for 7-10 minutes. Cylindrical hydrogels of 5 mm diameter and 1 mm height were formed. Hydrogels were swollen to equilibrium in sterile PBS before experimental use. For in vitro cell-based studies, hydrogels were secured to the bottom of 96 well plate with sterile vacuum grease. MGS and PEEK were secured to 96 well plate with GLUture (Penn Veterinary Supply) prior to seeding with cells.

Material Characterization: Water contact angle was measured using a goniometer (Ramé-Hart; Model 250-F1, Succasunna, NJ, USA). Each hydrogel was swelled to equilibrium and weighed, then freeze dried and weighed again to determine the mass swelling ratio. To quantify protein adsorption, each material was soaked in blood derived pooled human plasma (Innovative Research, IPLAWBNAC50ML) for 2 hours at 37° C., and then the plasma solution was removed by aspiration. Adsorbed plasma protein was removed from each material by soaking in 5% SDS in DI water for 1.5 hours on a shaker plate at 37° C. and quantified using a Pierce BCA protein assay kit (Cat. 23225). In brief, the samples were soaked in a BCA working reagent for five minutes and compared to a standard curve using known volumes of plasma. Samples were then cooled to room temperature and absorbance was measured at 562 nm. PEEK and MGS were compressed on a uniaxial test machine (MTS Eden Prairie, MN, USA), while PEG10 and PEG20 were subjected to unconfined compression at 10%/min (Bose LM0 Motor). Compressive modulus was calculated from the slope of the stress-strain curve between 10 and 15% strain.

Animals: TLR2^{-/-} and TLR4^{-/-}on the C57BL/6 background and C57BL/6J (Wildtype, Wt) mice were purchased from the Jackson Laboratories (Bar Harbor, ME) and bred in house. TLR2^{-/-} and TLR4^{-/-} were crossbred to create the double knockout (DKO) line. Mouse genotypes from tail biopsies were confirmed by Transnetyx (Cordova, TN).

Subcutaneous Implantation: Each material was implanted subcutaneously in mice, and the location of each material was randomly assigned. Each mouse received four implants, one above each shoulder and one above each hip. A blunt dissection technique was used to push the implant away from the initial incision to prevent any wound healing response interference. Explants and the surrounding tissue were removed 28 days after implantation. All animal protocols follow the NIH guidelines for care and use of laboratory animals and were approved by the Institutional Animal Care and Use Committee (IACUC) at the University of Colorado at Boulder.

Histology and Immunohistochemistry: Samples were fixed in 10% formalin, neutral buffered for 4 hours, then rinsed and stored at 4° C. in PBS until further processing. Fixed samples were deyhydrated, embedded in paraffin, and sectioned as 6 μm thick samples. PEEK and MGS were carefully removed from the surrounding tissue during embedding in paraffin. Sections were stained with Gömöri trichrome (Epredia), which stains collagen blue, muscle and cytoplasm red, and nuclei black. The inflammatory cell layer (ICL) and the fibrous capsule (FC) layer thickness were quantified using ImageJ. For each sample, three different sections were imaged, and three measurements were made for both the ICL and FC across each image and averaged to get a single value for each sample. All measurements were made on the dorsal side of the implant. Samples were also stained with hematoxylin and eosin (H&E) (Vector Labs), where nuclei are stained dark purple, and the cytoplasm and extracellular matrix are stained pink.

Immunohistochemistry was performed to identify macrophages by anti-Mac-3. First, samples were exposed to 3% hydrogen peroxide (Fisher) in methanol (Macron) for ten minutes and rinsed with PBS to block peroxidase. Then antigen retrieval was performed using Retrievagen A (BD Pharmingen), and samples were rinsed in PBS before permeabilizing and blocking non-specific binding with 10% normal goat serum (Invitrogen), 1% bovine serum albumin (BSA) (Sigma), and 0.025% Triton-X100 (Sigma) in PBS. Next, samples were soaked in primary antibody (rat anti-mouse CD107b, BD Pharmingen) at a 1:30 concentration in 1% BSA in PBS overnight at 4° C. Next, samples were rinsed with PBS and the secondary antibody (Biotin mouse anti-rat IgG, BD Pharmingen) was applied at a 1:200 concentration in 1% BSA in PBS for thirty minutes at room temperature. Samples were then rinsed and treated with streptavidin-HRP (BD Pharmingen) for thirty minutes, then rinsed and treated with DAB (Fisher). Lastly, samples were counterstained with methyl green for five minutes at 60° C. before dehydrating and mounting with Citramount (Polysciences).

Neutrophil isolation and biomaterial seeding: Mice were euthanized via CO₂ asphyxiation and cervical dislocation, then both femurs and tibias were collected and stored in RPMI 1640 (Gibco). Each bone was flushed with RPMI 1640 media (10% FBS, 2 mM EDTA (Invitrogen)) and the bone marrow was pooled based on sex and strain. Red blood cells were lysed with 0.2% NaCl and 1.6% NaCl (Fisher) and the remaining cells were washed twice with PBS containing 2 mM EDTA. The neutrophils were isolated following standard protocols via density gradient with histopaque 1119 (Sigma) and histopaque 1077 (Sigma). The neutrophils were collected, washed, and plated in a non-TC treated flask with RPMI 1640 media for 30 minutes. Neutrophils do not attach to non-TC treated plates, but other cells do, during this time frame. The unattached cells were collected. The isolated neutrophil population was 74% pure, confirmed with flow cytometry. Immediately after isolation the neutrophils were counted and plated at 100,000 cells per well on TCPS or on a biomaterial secured to the bottom of a 96 well plate. The conditions were: PEEK, MGS, PEG10, and PEG20. Each biomaterial was exposed to human plasma immediately before beginning the neutrophil isolation, resulting in a total exposure time of 3 hours. Excess plasma was removed from the biomaterials, and freshly isolated neutrophils were seeded in RPMI 1640 with 0.2% ITS for 6 h on each biomaterial and TCPS. TCPS served as a control and was not exposed to plasma.

Neutrophil cell attachment: To quantify neutrophil attachment 6 hours post-seeding, the medium was removed, and cells were immediately lysed with 0.05% Triton-X100 in DI water followed by three freeze thaw cycles. The cell lysate was stored at-70° C. Neutrophil cell lysates were thawed on ice and combined with Hoescht assay dye solution in the dark, then fluorescence was measured at 360 nm excitation/465 nm emission. Results were compared to a standard curve containing known concentrations of calf thymus DNA in 0.05% Triton-X100.

Macrophage isolation and biomaterial seeding: Peritoneal macrophages were isolated from each mouse strain. Briefly, 1.5 ml of sterile 4% Brewer's Thioglycolate (BD Pharmingen) was injected into the intraperitoneal region of mice from each strain. Each mouse was euthanized 3 days later via CO₂ asphyxiation and cervical dislocation. The abdominal cavity was washed with 10 ml of sterile PBS to collect the recruited macrophages. The macrophages were then counted and plated on non-TC treated flask for 24 hours in IMDM media (10% fetal bovine serum (FBS), 1% penicillin/streptomycin (P/S)) before use. Macrophages were seeded at 100,000 cells per well on each biomaterial in a 96 well plate. The conditions are as follows: Macrophages were either naïve or primed to a pro-inflammatory phenotype at the time of seeding. To prime macrophages towards a pro-inflammatory polarization state, TLR2^{-/-} macrophages were stimulated with 1 ng ml⁻¹ LPS (TLR4 agonist, Invitrogen), TLR4^{-/-} macrophages were stimulated with 1 ng ml⁻¹ PAM3CSK4 (TLR2 agonist, Invitrogen), and DKO macrophages were stimulated with 0.5 ng ml⁻¹ LPS and 0.5 ng ml⁻¹ PAM3CSK4. Prior to cell seeding, each biomaterial was soaked in human plasma for 2 h excess plasma was removed, and the cells were seeded in serum-free IMDM media supplemented with 0.2% ITS without or with pro-inflammatory primer. After 4 or 24 h the medium was collected and cytokine production analyzed by ELISA.

Protein quantification: For neutrophils, the medium was removed 6 h post-seeding and stored at-70° C. for ELISA. Neutrophil elastase (NE) and myeloperoxidase (MPO) Duoset ELISA kits were used (R&D systems). For macrophages, the medium was removed at 4 or 24 h and stored at-70° C. for ELISAs. IL-6, TNF-α, and IL-18 Duoset ELISA kits were used (R&D Systems).

Statistical Analysis: The in vivo studies were performed with a minimum of six biological replicates. The in vitro studies were performed with a minimum of four replicates. Each dataset was tested for normality using the Shapiro-Wilk test and for homogeneity of variance using the Levene's Test. A one-way Analysis of Variance (ANOVA) was performed followed by Tukey's post hoc analysis for pairwise comparisons. When datasets did not meet the ANOVA requirements, a Kruskal-Wallis test was performed followed by Mann-Whitley test for pairwise comparisons. Statistical significance was set at p<0.05.

Part 2—Stimuli-Responsive Delivery of a Small Molecule Inhibitor of MYD88 from Biomaterials to Attenuate Inflammation

Stimuli-responsive biomaterials are ideal candidates for the delivery of anti-inflammatory small molecules in the treatment of chronic inflammation. For example, the upregulation of matrix metalloproteinases in the context of inflammation could be utilized in the design of enzyme-cleavable therapeutic release mechanisms, and the phagocytic environment present in macrophages could reduce disulfide bonds or encourage hydrolytic degradation to free a payload. It is shown herein that the myeloid differentiation primary response protein 88 (MyD88) inhibitor T6167923 can be employed as a releasable molecule for its ability to attenuate inflammation that progresses via Toll-like and IL-1 receptor pathways. The commercially-available free inhibitor as well as a custom-synthesized version containing a sulfhydryl group for synthetic purposes demonstrated attenuation of IL-6 pro-inflammatory cytokine production in RAW 264.7 macrophages in a dose-dependent manner. PAM3CSK4 was used to stimulate an inflammatory response through a TLR2 dependent mechanism. At a dose of 100 μM, the T6167923 (Br) and T6167923 (SH) attenuated PAM3CSK4-stimulated IL-6 production by 89% and 58%, respectively. To deliver the T6167923 to macrophages, a stimuli-responsive biomaterial platform based on macrophage metalloelastase (MMP12) was employed. A poly(ethylene glycol) (PEG) hydrogel with an enzyme-cleavable peptide sequence linking the inhibitor to the gel was of interest for subcutaneous implantation to address the inflammation in tissues, such as through the foreign body response, and reduce the inflammatory cell layer thickness. First, the secretion of MMP-12 by the cells was verified, then peptides were immobilized in a hydrogel matrix and exposed to the enzyme in solution to confirm that the chosen sequences were cleaved by the enzyme. A release curve was generated which indicated that both peptide sequences had significant cleavage over time, with the shorter of the two sequences released at a slightly faster rate. When incorporated into a hydrogel via the MMP-12 sensitive linker, the inhibitor-tethered hydrogels led to a 50% decrease in the inflammatory cell layer thickness relative to the non-therapeutic PEG hydrogel control. In summary, this demonstrates a promising novel therapeutic hydrogel that is capable of attenuating inflammation when triggered by macrophage metalloelastase.

MyD88 is an intracellular adapter protein that is required for TLR2 signaling. TLR4 signaling has two pathways, one uses MyD88 and the other uses the adapter protein TRIF. The MyD88 pathway leads to upregulation of many pro-inflammatory cytokines (e.g., interleukin-6, tumor necrosis factor alpha, and interleukin 1b). The examples below describe a novel approach to attach a modified version of an MyD88 inhibitor that allows it to be attached to a biomaterial and subsequently released. A similar approach could be employed with an immobilized TRIF inhibitor in place of or in addition to the MyD88 inhibitor, thus targeting both of the pathways in the TLR4 as needed. A similar approach could be done with any inhibitor of these pathways. It is also shown through immobilization into a hydrogel. However, a similar approach could be done with any material. As one example, any biomaterial surface could be treated with a silane to produce reactive thiols, acrylates, etc. which can then be used to attach the inhibitor with a PEG linker and a degradable linker (e.g., MMP12-sensitive used in the attached document) to the surface of an implant.

Inflammation is an important biological response coordinated by innate immune cells. It plays a paramount role in controlling infection and directing tissue repair mechanisms. The immune response starts with recognition of foreign stimuli by myeloid cells such as macrophages and progresses through complex pathways that induce production of pro-inflammatory cytokines and other chemokines that recruit additional immune cells to the site of infection or injury. In acute inflammation, this cascade leads to removal of the foreign stimuli, resolution of inflammation, and return to homeostasis. However, chronic inflammation causes pathological outcomes, such as inflammation-associated progressive disease states and fibrosis. Similar processes occur when a biomaterial is implanted into the body. When the innate system is unable to remove a foreign biomaterial, the material is often walled off by a fibrous capsule and chronic inflammation ensues as long as the foreign material remains, which can be problematic for implantable medical devices.

Biomaterial-based strategies have been developed for targeting inflammation with controlled delivery of anti-inflammatory drugs to support the healing process. The mechanisms of delivery include controlled release through degradation of the material or by an endogenous stimuli, such as redox potential, pH, or enzymes that triggers release in response to changes in the local environment. Microparticles have been used to encapsulate drugs that are then either used directly depending on the application or subsequently encapsulated into another biomaterial. Poly (lactic-co-glycolic) acid (PLGA) microparticles can deliver anti-inflammatory drugs, such as dexamethasone, mediated through polymer degradation. For example, PLGA loaded with dexamethasone attenuated inflammation in lipopolysaccharide-stimulated macrophages in vitro and have been used to treat acute respiratory distress syndrome in a rat model. Loading dexamethasone-containing PLGA microparticles into a hydrogel demonstrated prolonged delivery of drug compared to free microparticles or free drug. A non-healing wound infection application utilized PLGA microparticles loaded with vascular endothelial growth factor for tissue growth and dispersed the particles within injectable, pH-sensitive chitosan/hyaluronic acid hydrogels containing the antibiotic vancomycin for infection resolution to create a staged therapeutic release. An example of exogenous stimuli for controlled release is covalently linking a drug via a peptide-tether that is sensitive to enzymes such as matrix metalloproteinases (MMPs). MMPs catalyze the hydrolysis of peptidic bonds and tend to be overexpressed in the context of inflammation and disease.

Myeloid differentiation primary response gene 88 (MyD88) is an adaptor protein that is central to inflammation. MyD88 is required for activation of signaling by the Interleukin-1 receptor (IL-1R) family and nearly all the Toll-like receptors (TLRs). Several prominent pro-inflammatory cytokines signal through the IL-1R family, including IL-1, IL-18, and IL-33. TLRs recognize many different pathogen-associated molecular patterns (PAMPs) and damage-associated molecule patterns (DAMPs) to initiate an inflammatory response. MyD88 knockout mouse models have demonstrated its central role in mediating airway, nephritic, and lipopolysaccharide-induced inflammation; pulmonary, renal, and hepatic fibrosis; and the foreign body response to implantable biomaterials. As such, MyD88 is a promising therapeutic target for treating chronic inflammation because of its essential role in mediating inflammation in response to several cell surface receptors that induce pro-inflammatory cytokine production.

Several small molecule peptidomimetic and synthetic inhibitors of MyD88 have been developed as potential therapeutics. These molecules disrupt MyD88-mediated signaling by binding to the Toll-interleukin receptor-domain (TIR) of MyD88, preventing its binding and formation of the homodimer that is necessary to activate signaling. In vitro studies have shown a dose-dependent reduction in the expression of pro-inflammatory cytokines such as interleukin-6 (IL-6), tumor necrosis factor-α (TNF-α), and interleukin-1B (IL-1) when stimulated cells are treated with a MyD88 inhibitor of MyD88 inhibitors have been demonstrated in vivo to attenuate MyD88-dependent immune responses in mice with induced colitic, neurological, vascular, renal, cardiac, pulmonary, and hepatic inflammatory conditions and infection. Thus, MyD88 inhibitors are promising therapeutics to treat the wide range of MyD88-mediated inflammatory diseases.

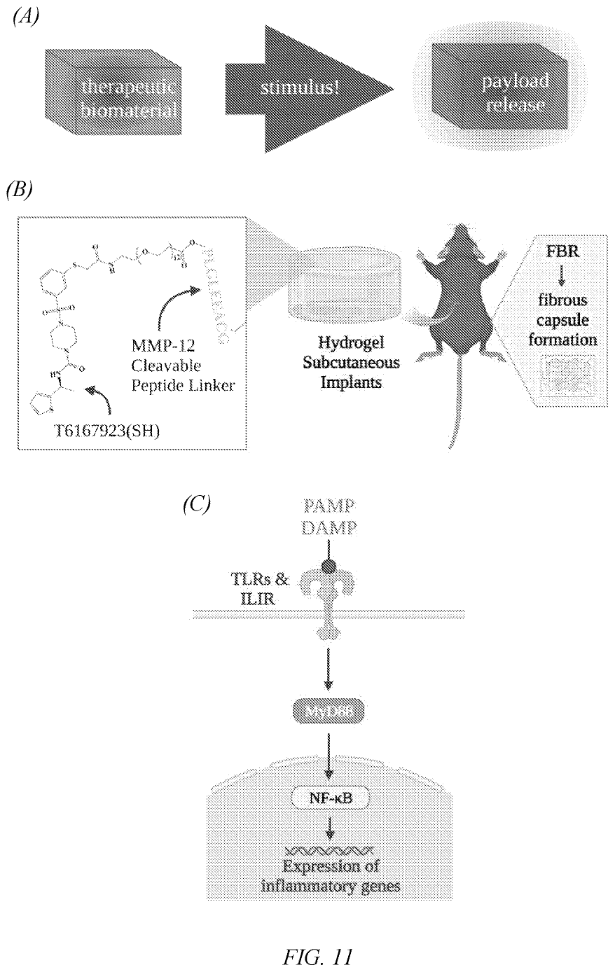

This work investigates immobilizing the small molecule MyD88 inhibitor T6167923 to a biomaterial as a potential biomaterial-based therapeutic approach to attenuate inflammation. This work examines tethering T6167923 to a biomaterial via a degradable linker whose chemistry is designed to limit its release to a targeted environment (FIG. 11). To achieve immobilization, T6167923 was first custom synthesized with a sulfhydryl group replacing the bromine group. The effectiveness of the custom synthesized T6167923 (SH) was first compared to the commercially available T6167923 (Br) in attenuating inflamed RAW 264.7 macrophages. To demonstrate the potential of this biomaterial-based therapeutic approach and show that controlled release of T6167923 attenuates the inflammatory response of immune cells in vivo, an application focused on the foreign body response was developed (FIG. 11B). A significant reduction in inflammatory cell accumulation in MyD88 knockout mice is observed. In the present approach, T6167923 was linked to a biomaterial via a matrix metalloproteinase sensitive crosslinker. For both applications, the immobilization strategy and release of T6167923 were characterized; in vitro studies were performed with Pam3CSK4-treated RAW 264.7 macrophages, a TLR2 agonist that mediates inflammation solely through MyD88; and the effectiveness of the therapeutic biomaterial was assessed in vitro and in vivo in a lung injury model and a subcutaneous biomaterial implantation model in mice.

It is shown herein that an enzyme-cleavable PEG hydrogel delivery system can release a small molecule therapeutic in response to a stimulant. Addressing chronic inflammation and the FBR, the MyD88 inhibitor T6167923 was selected and its ability to attenuate pro-inflammatory IL-6 cytokine production in a dose-dependent manner. A peptide sequence cleavable by MMP-12 was chosen because of the enzyme's role in inflammation and disease and its prolific secretion by macrophages. Subcutaneous implantation of the therapeutic PEG hydrogel led to a 50% reduction in inflammatory cell layer thickness relative to control.

In certain aspects the present invention provides a new therapeutic biomaterial that targets MyD88, a central mediator of inflammation. Release of a small molecule MyD88 inhibitor was designed to respond to MMP-12, which is produced by macrophages. Overall, it is shown that this stimuli responsive material is able to attenuate the inflammatory response associated with the foreign body response.

T6167923 (Br) and T6167923 (SH) attenuated TLR2-mediated inflammation in RAW 264.7 macrophages. PAM3CSK4 was used to induce an inflammatory response by activation of TLR2. Attenuation of the inflammatory response by the free inhibitors was dose dependent. In the current study, the commercially-available T6167923 (Br) was more effective than the custom T6167923 (SH) at attenuating the inflammatory response and over a wider range of treatment concentrations. It is unlikely that reagent contaminants were responsible for this phenomenon given that the purity of the T6167923 (SH) was reported to be 95% by the manufacturer. Rather, the lesser ability of T6167923 (SH) to attenuate inflammation may be due to the presence of the sulfhydryl group, which could lead to interactions with sulfhydryl groups present on the cell membrane and impact the ability of the small molecule to diffuse across the membrane. Previous studies have shown that thiolated molecules such as PEG dithiol and cell penetrating peptides with a sulfhydryl group are able to interact with sulfhydryl groups on cell membranes via formation of disulfide bonds. The latter study showed that the thiolated molecules, despite their immobilization to the cell membrane via disulfide bonds, were eventually internalized. The present study explored only one treatment timing; thus, it is possible that with longer treatment times, the T6167923 (SH) may have shown improved effectiveness.

MMP-12 enzyme production by RAW 264.7 macrophages was dependent on the type of culture medium as well as presence of TLR2 stimulus. MMP-12, also known as macrophage metalloproteinase, is most plentifully secreted by macrophages; this allows for a more cell-specific targeted release. RAW 264.7 cells produced less MMP-12 in serum-containing medium compared to serum-free medium, which may be attributed to the presence of MMP-regulating proteins in the serum such as growth factors and hormones that regulate MMP synthesis. In addition, serum contains MMP inhibitors such as the broad-spectrum protease inhibitor alpha-2-macroglobulin. Thus, serum-free media was preferred to avoid confounding effects with MMP inhibitors. MMP-12 production was also attenuated by TLR2 induction. MMP-12 has myriad functions in both physiological and pathological conditions. Important to the findings presented here, MMP-12 is involved in wound healing and anti-inflammatory pathways which provides context for why MMP-12 secretion was lower under PAM3CSK4-stimulated conditions than the unstimulated comparison.

Having demonstrated that RAW macrophages secrete MMP-12, it was assessed whether MMP-12 could induce release of T6167923 from a therapeutic hydrogel. To do this, a tethering strategy was designed with a PEG linker to reduce steric hindrance and allow access of the MMP12 molecule to bind to the peptide. Both peptide sequences were cleaved by MMP-12. There was a slight, but significant increase in release for the eleven-residue sequence over the twelve-residue sequence, which is attributed to the amino acid sequence. Altering the amino acids adjacent to the degradable bond can affect the degradation kinetics. Therefore, further manipulation of the cleavable peptide's amino acid sequence could offer a greater ability to tune the degradation kinetics.

MMP-12 induced release of T6167923 from the therapeutic hydrogel, attenuated inflammation in the foreign body response to PEG hydrogels. MyD88 is a primary driver of the FBR to implantable biomaterials. Moreover, MMP-12 expression by macrophages during the FBR has been observed. The current subcutaneous implantation study shows that it is possible to attenuate inflammation through the release of T6167923, indicating that enough MMP-12 is produced at the site of FBR to cleave the peptide linker and free the therapeutic. This is an important finding that highlights the ability of the biomaterial system developed herein to provide specific, stimuli-responsive release that utilizes the cues present at the location of delivery. Future studies could focus on tuning the release rate of the inhibitor to control the release profile, such that clinically relevant treatment timelines could be tailored.

### Example 8—Attenuation of RAW 264.7 Macrophage IL-6 Production by Free Inhibitor

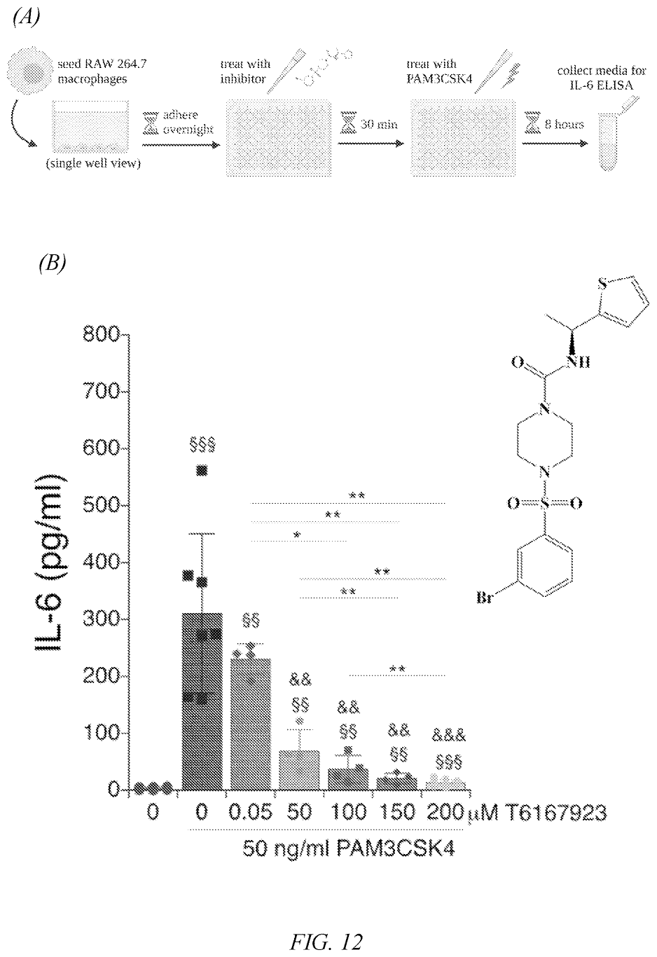

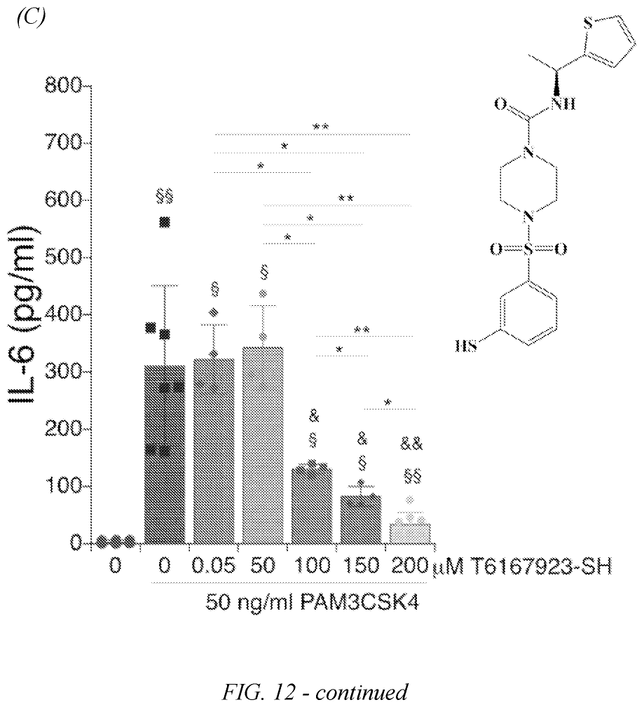

The ability of free MyD88 inhibitor T6167923 (Br) or T6167923 (SH) in solution to attenuate pro-inflammatory cytokine production by macrophages treated with PAM3CSK4 was assessed (FIG. 12A). When macrophages were treated with PAM3CSK4, IL-6 production increased (p<0.01) by 3-fold. With the treatment of T6167923 (Br), a dose-dependent response was observed (FIG. 12B). Specifically, IL-6 levels were significantly lower (p<0.01) for cells treated with 50-200 μM inhibitor compared to the untreated PAM3CSK4-stimulated cells. With treatment of T6167923 (SH), a dose-dependent response was also observed (FIG. 12C). However, IL-6 production was significantly lower over a narrower range of concentrations at 100-200 μM. Comparing the two inhibitors at 100 μM, T6167923 (Br) had attenuated IL-6 production by 89%, while T6167923 (SH) had attenuated IL-6 product by 58%.

### Example 9-Synthesis of T6167923 (SH) Linker

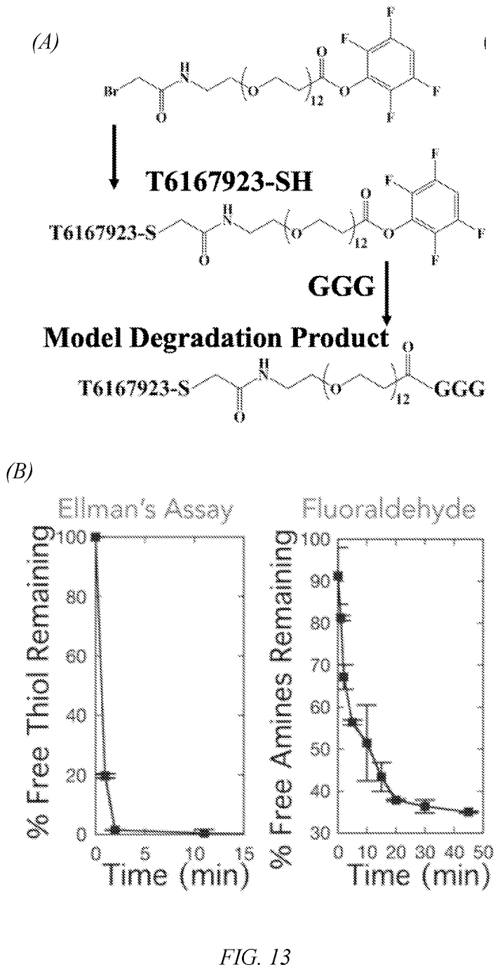

A stimuli-responsive biomaterial that releases the T6167923 upon degradation by MMP was designed with an MMP-sensitive peptide and PEG linker to improve the water solubility of the inhibitor once released. The released inhibitor will have a PEG linker and a short peptide sequence after MMP degradation. To confirm the synthesis, we utilized a model peptide triglycine. The thiol on T6167923 (SH) was reacted with bromoacetamido-dPEG12-TFP ester at the bromoacetamido site. Next, a model peptide triglycine was conjugated to the PEG at the TFP ester. The product represents the synthesis used to attach the MMP-12 sensitive peptide to the inhibitor. The reaction scheme is shown in FIG. 13A. Ellman's assay was used to track progress of the first reaction step; consumption of free sulfhydryl groups was monitored over time, with greater than 95% of the sulfhydryl groups reacted within the first three minutes (FIG. 13B). Consumption of amines was characterized by Fluoraldehyde assay and showed that less than 40% remained unreacted after 50 minutes of reaction time (FIG. 13B). These results confirm the ability to attach the inhibitor to a peptide via a PEG linker.

### Example 10-MMP-12 Production and Activity

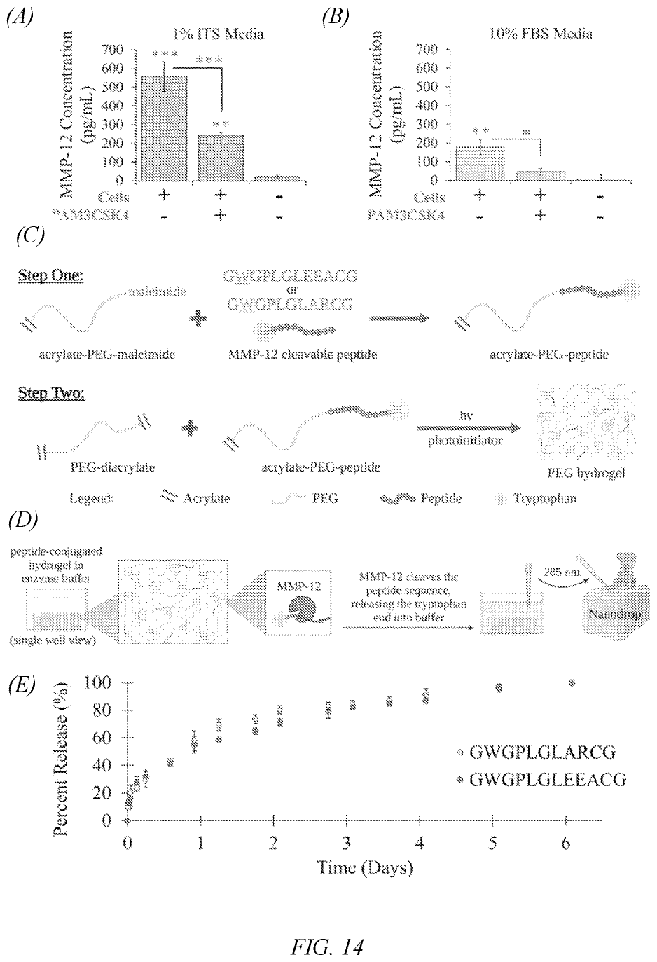

RAW 264.7 macrophages were cultured in serum-free (1% ITS) media (FIG. 14A) or serum-containing medium (10% FBS) (FIG. 14(B)) and treated or not with PAM3CSK4 (50 ng/ml), then their secretion of MMP-12 over the 8-hour period was evaluated by ELISA. Macrophages with (p<0.01) and without (p<0.001) PAM3CSK4 stimulation secreted MMP-12 when cultured in the serum-free media, with average levels of 245 and 556 μg/mL, respectively. In serum-containing media, the production of MMP-12 was on average 178 μg MMP-12/mL (p<0.01 relative to blank) in unstimulated cells. With stimulation, MMP-12 levels were not statistically different to the blank (no cells), indicating that in serum-containing medium with PAM3CSK4, MMP-12 production was inhibited. Unstimulated cells in the same media secreted MMP-12. MMP-12 production by the cells was higher in ITS media than in FBS media.

To test the cleavability of the chosen peptides by MMP-12, the sequences were modified with tryptophan residues, tethered to PEG hydrogels at 5 μM concentration, and exposed to 5 nM MMP-12 in buffer. First, the maleimide of acrylate-PEG-maleimide (MW 3,400 g/mol) was reacted with the cysteine residue contained in the peptide sequence to create a PEG-conjugated peptide with tryptophan at one end and an acrylate group at the other. This product was then combined with PEGDA monomer and photoinitiator in PBS and photopolymerized, yielding a gel (FIG. 14C). The release of the tryptophan-containing cleaved portion of the sequence was monitored by measuring buffer absorbance at 285 nm over time (FIG. 14D). Both peptide sequences were cleaved by the MMP-12, as shown in the plot of percent release versus time (p<0.001), with the shorter peptide sequence (GWGPLGLARCG [SEQ ID NO. 1]) cleaved at a slightly faster rate (p<0.001) than the longer peptide sequence (GWGPLGLEEACG [SEQ ID NO. 2]) (FIG. 14E).

### Example 11—Hydrogel-Facilitated Delivery of T6167923

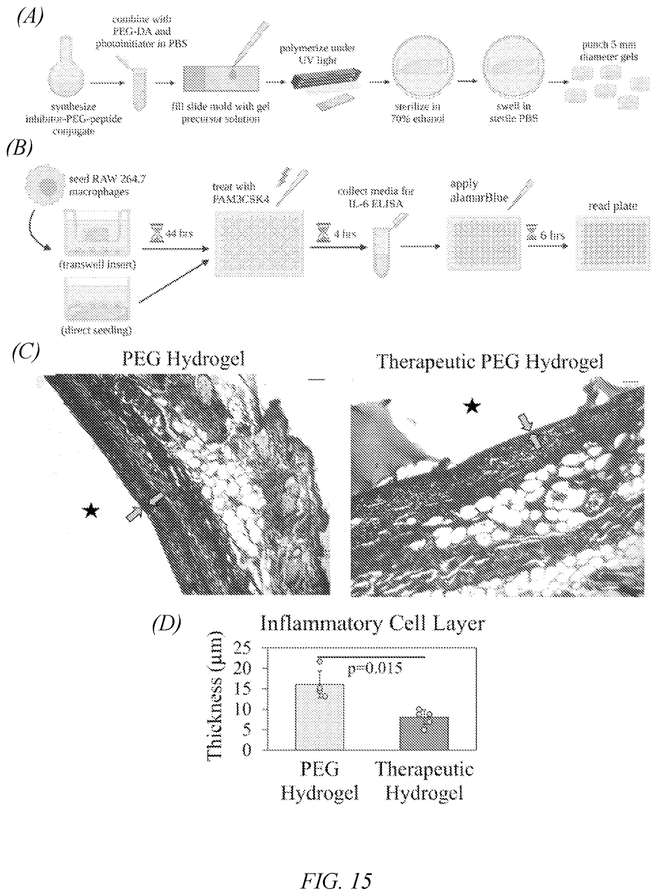

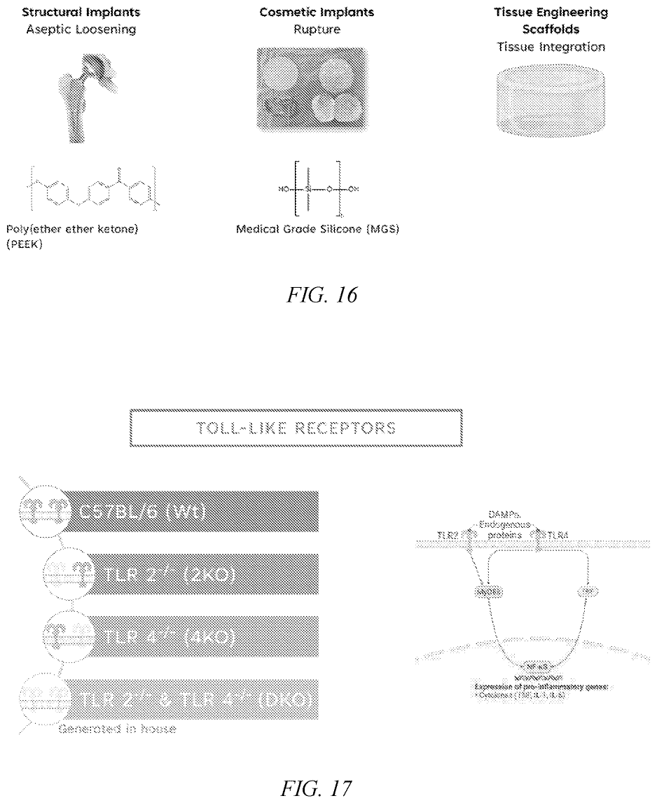

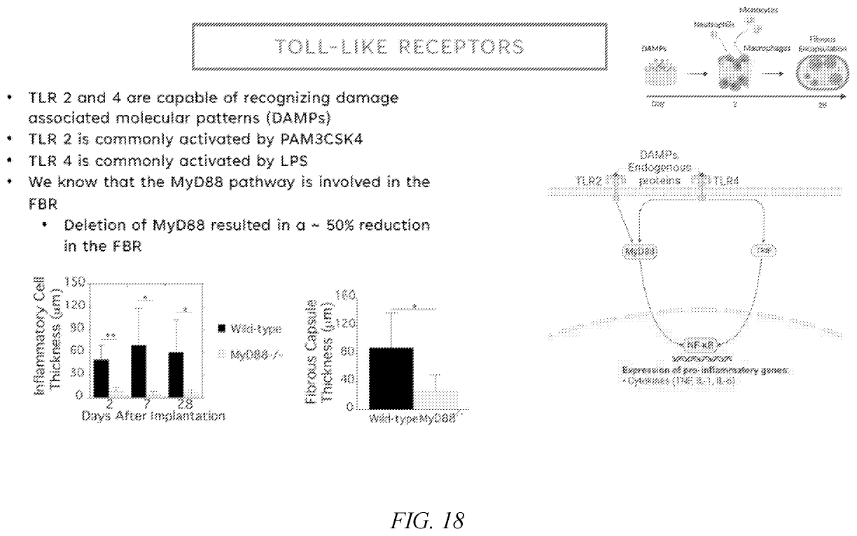

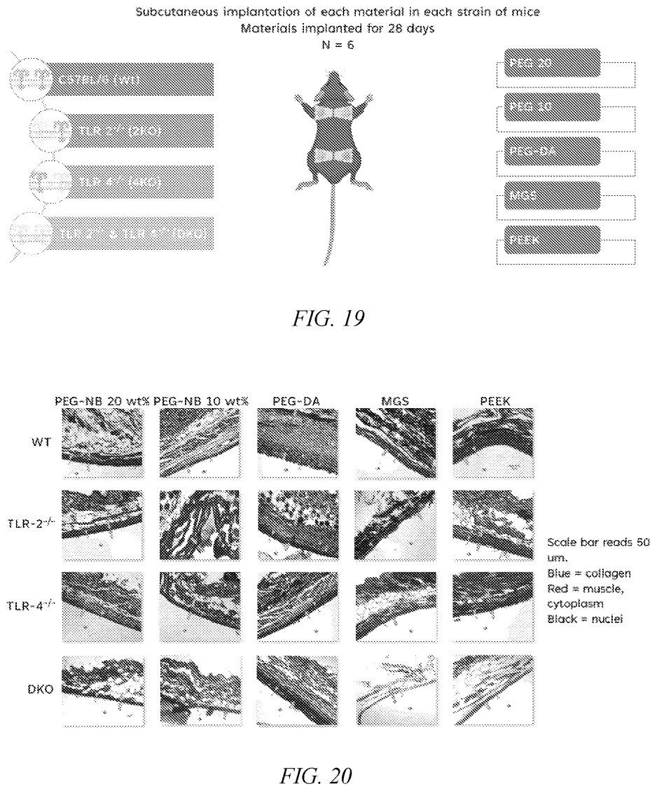

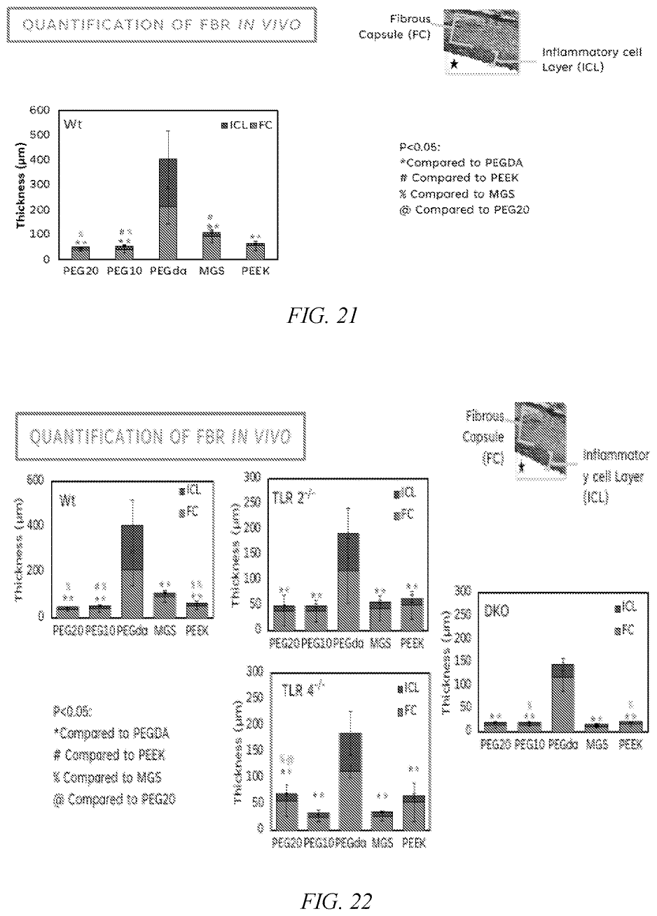

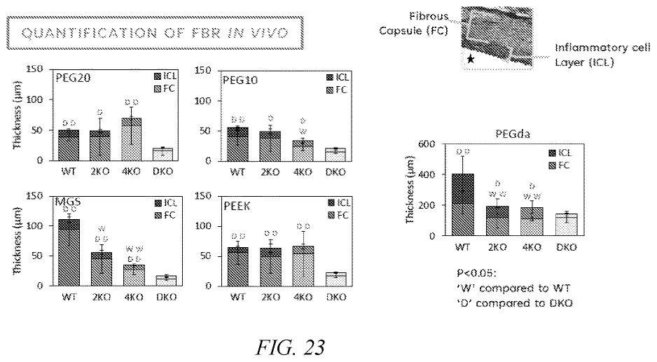

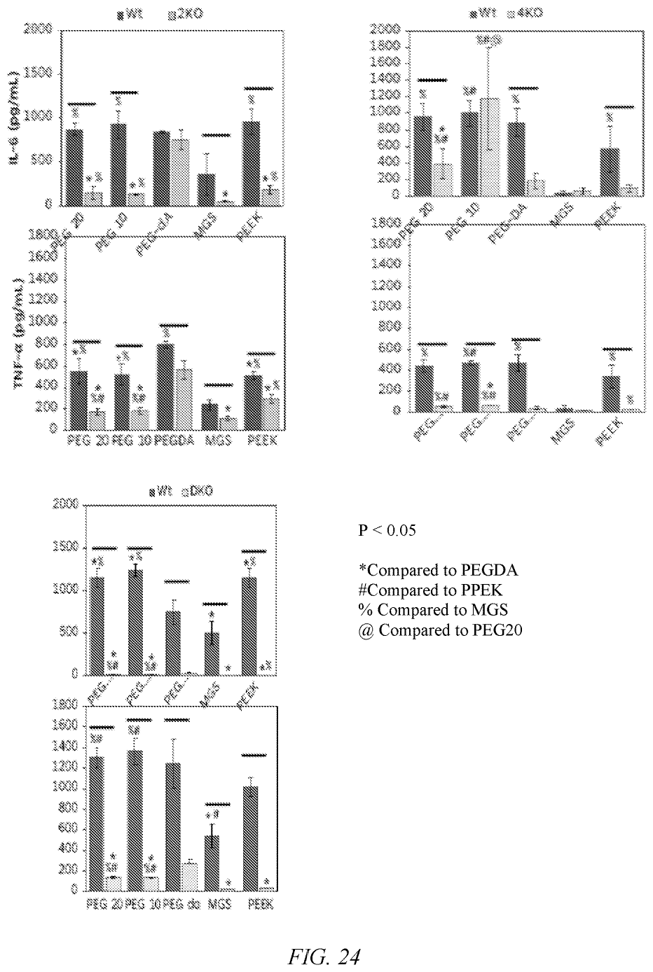

To synthesize the therapeutic PEG hydrogels, bromoacetamido-dPEG12-TFP ester was first reacted with T6167923 (SH) at the bromoacetamido site then subsequently with the MMP-12 cleavable peptide sequence PLGLEEACG [SEQ ID NO. 3] at the TFP ester leaving group, following a similar protocol outlined in FIG. 14. This molecule was tethered into a PEG hydrogel by conjugating it to acrylate-PEG-maleimide (3,400 g/mol) via the cysteine side chain within the peptide sequence, followed by photopolymerization with PEGDA monomers. PEG hydrogel controls were synthesized from PEGDA monomer only. The overall approach is outlined in FIG. 15A.

The therapeutic and control PEG hydrogels were implanted subcutaneously for seven days. Histological analysis by Masson's Trichrome stain shows a layer of immune cells present at the interface of the implant on the dorsal side, shown by the arrow in FIG. 15C. A thicker layer of immune cells is observed with the control PEG hydrogel. Although day 7 is in the infancy of fibrous capsule formation, a more organized collagenous matrix is observed adjacent to the inflammatory cell layer when compared to the therapeutic hydrogel. The thickness of the inflammatory cell layer was quantified from the stained images (FIG. 15D). The inflammatory cell layer adjacent to the therapeutic PEG hydrogel implants was reduced by 50% (p=0.015) relative to control hydrogels.

### Example 12—Materials and Methods—Part 2

Materials: RAW 264.7 macrophages were obtained from ATCC. Mouse MMP12 SimpleStep ELISA kit was purchased from Abcam. T6167923 was procured from Aobious, Inc. Fetal bovine serum (FBS) was procured from Atlanta Biologics. Poly(ethylene glycol) (PEG) diacrylate (PEGDA, 3,400 g/mol) and poly(ethylene glycol) dithiol (PEGDT 3,400 g/mol) were purchased from Biopharma PEG Scientific, Inc. 2-Morpholinoethanesulfonic acid (MES) was obtained from Chem-Impex International, Inc. Irgacure 2959 was purchased from Ciba Specialty Chemicals, Inc. Dulbecco's phosphate buffered saline (DPBS), Dulbecco's Modified Eagle's Medium (DMEM), glutagro, ITS Premix, and penicillin/streptomycin were purchased from Corning. T6167923 (SH) was custom synthesized by Enamine Ltd. Poly(ethylene glycol) 3000 MW was obtained from Fluka. MMP-12 cleavable peptides (GWGPLGLARCG [SEQ ID NO. 1], GWGPLGLEEACG [SEQ ID NO. 2], and PLGLEEACG [SEQ ID NO. 3]) were custom synthesized by GenScript. Transwell inserts were purchased from Greiner Bio-One. LPS from E. coli 0111: B4 (LPS-EB) and PAM3CSK4 were procured from InvivoGen. Acrylate-PEG-Maleimide, 3,400 g/mol was purchased from Laysan Bio. Bromoacetamido-dPEG®12-TFP ester was purchased from Quanta BioDesign. Mouse IL-6 and TNF-α Duo Set ELISA kits and Recombinant Human MMP-12 Protein, Carrier Free were obtained from R&D Systems. 5,5′-Dithiobis-(2-Nitrobenzoic Acid) (DTNB; Ellman's Reagent); alamarBlue™ Cell Viability Reagent; calcium chloride anhydrous, ethylenediaminetetraacetic acid (EDTA); Fluoraldehyde™ o-Phthaldialdehyde Reagent Solution (OPA); L-tryptophan; methanol; N,N-dimethylformamide (DMF), potassium phosphate, dibasic; sodium bicarbonate; sodium chloride; and zinc sulfate heptahydrate were purchased from Thermo Fisher Scientific. Dipentaerythritol hexa-3-mercaptopropionate (DPHMP) was obtained from Tokyo Chemical Industry. Anhydrous dimethyl sulfoxide (DMSO) was procured from VWR International.

Inhibitor Preparation and Synthesis: 200 μM stock solutions of T6167923 (Br) and T6167923 (SH) were prepared by first dissolving the inhibitors in 100 μL anhydrous DMSO, then adding dropwise to 9.9 mL culture medium and sterile filtering. The stock solution was then serially diluted in additional cell culture medium to make treatment concentrations. The inhibitor-PEG-peptide molecule was synthesized as follows. Bromoacetamido-dPEG12-TFP ester (36 mg) was added to 400 μL anhydrous DMSO and stirred in an amber vial to dissolve. T6167923 (SH) (17.65 mg) was dissolved in 100 μL anhydrous DMSO before being added dropwise to the amber vial. After 20 minutes of stirring, 1.5 mL of 50 mM sodium bicarbonate buffer, pH 8.4 was slowly added to the vial and the reaction was continued for an additional 20 minutes. Triglycine (8.6 mg) was combined with 2 mL of 50 mM sodium bicarbonate buffer, pH 8.4, then added dropwise to the reaction mixture in the amber vial with stirring. The reaction progressed overnight, then the product was purified by dialysis with 100-500 MWCO tubing against DI water. The reaction was monitored by consumption of sulfhydryls and primary amines. The product was sterile filtered and then lyophilized.

Sulfhydryl Characterization: The consumption of inhibitor free sulfhydryl groups in the reaction of T6167923 (SH) with bromoacetamido-dPEG12-TFP ester was monitored over time and immediately characterized by Ellman's assay. Ellman's reagent (4 mg) was combined with 1 mL reaction buffer (0.1M sodium phosphate, pH 8.0, 1 mM EDTA). 50 μL of this solution was added to 2.5 mL reaction buffer for each sample, then 300 μL of sample from the reaction diluted in sodium bicarbonate buffer was added. 200 μL of this solution was transferred to a clear, flat bottom 96-well plate and the absorbance was read at 412 nm. A standard curve was prepared with free T6167923 (SH) in 50 mM sodium bicarbonate buffer, pH 8.4, with 0.75% anhydrous DMSO by volume for comparison to determine sulfhydryl concentration during reaction progress.

Amine Characterization: The consumption of primary amines in the reaction of peptide with bromoacetamido-dPEG12-TFP ester was monitored over time using Fluoraldehyde™ o-Phthaldialdehyde Reagent Solution (OPA). The bromoacetamido on the bromoacetamido-dPEG12-TFP ester (10 mg) was first reacted with thiophenol (1.3 μL), a model molecule, in 1 mL of 50 mM sodium bicarbonate buffer for half an hour. Then triglycine (2.3 mg) dissolved in anhydrous DMSO (50 μL) was added and the reaction was immediately monitored. Aliquots were taken and combined with Fluoraldehyde, then fluorescence was read at λₑₓ=340 nm and λₑₘ=455 nm within 5 minutes. A standard curve was prepared with triglycine in 50 mM sodium bicarbonate buffer, pH 8.4, with 0.75% anhydrous DMSO by volume for determination of free amine concentration over the course of the reaction.

Hydrogel Preparation: PEG control hydrogels were synthesized by combining PEGDA (100 mg) with DPBS (500 μL) and Irgacure 2959 photoinitiator (6 mg/mL in DPBS). The precursor solution was applied between glass slides with a 1 mm spacer, then polymerized under UV light for 45 minutes to make a 20 wt % hydrogel material. Therapeutic PEG hydrogels were created by first synthesizing the inhibitor-dPEG12-peptide conjugate and then photopolymerizing it into a hydrogel with PEGDA. Bromoacetamido-dPEG12-TFP ester (38.2 mg) was dissolved in 50 mM sodium bicarbonate buffer, pH 8.4 (2 mL). T6167923 (SH) (18.5 mg) was vortexed into anhydrous DMSO (0.2 mL) then added dropwise to the reaction mixture with stirring. An additional 1.8 mL sodium bicarbonate buffer were added in the same manner. After 30 minutes, sodium bicarbonate buffer (32 mL) was transferred to a round bottom flask and MMP-12 cleavable peptide (PLGLEEACG [SEQ ID NO. 3], 40 mg) dissolved in anhydrous DMSO (4 mL) was added dropwise with stirring. The reaction was continued for 24 hours, then the reaction mixture was purified by dialysis with 100-500 MWCO tubing against DI water prior to lyophilization. The product (25.1 mg) was combined with acrylate-PEG-maleimide (3,400 g/mol, 34 mg) in an amber vial containing 500 μL DPBS and stirred for half an hour. PEGDA (100 mg) was then added to the reaction mixture with Irgacure 2959 photoinitiator (6 mg/mL in DPBS). The gel precursor was pipetted between glass slides with a 1 mL spacer and photopolymerized under UV light for 45 minutes. Hydrogels for in vitro and in vivo applications were processed by sterilizing them in 70% ethanol for at least 24 hours before rinsing and swelling in sterile DPBS. The swollen hydrogel sheets were punched to 5 mm diameter under sterile conditions and stored in sterile DPBS prior to use in cell culture or surgery.

For the release studies, acrylate-PEG-maleimide (6.8 mg) was reacted with peptide (GWGPLGLEEACG [SEQ ID NO. 2] or GWGPLGLARCG [SEQ ID NO. 1], 5 mM) in 500 μL dry DMF for 30 minutes with stirring. PEGDA (100 mg) was then added to the reaction mixture and vortexed to combine. Irgacure 2959 was added as photoinitiator in dry DMF (6 mg/mL). This precursor solution was gelled between glass slides with a 1 mm spacer under UV light for 45 minutes. The resulting 20 wt % hydrogel was soaked in ultrapure water to rinse away DMF followed by additional rinses in TCNB assay buffer (50 mM Tris, 10 mM CaCl₂, 150 mM NaCl, 0.05% (w/v) Brij-35, pH 7.5).

Tryptophan Release: Peptide-conjugated hydrogels swollen in TCNB assay buffer were punched to 5 mm diameter and placed in wells of a 96-well clear, flat bottom well plate (n=4 gels for each peptide). Human MMP-12 was reconstituted in 10 mM MES, 0.1 M NaCl, 100 μM CaCl₂, 0.1% (w/v) CHAPS, 1 μM ZnSO₄, 0.1% (w/v) PEG, pH 6.0 buffer to 0.25 mg/mL. It was then diluted to 50 μg/mL with TCNB assay buffer and activated by adding 100 mM APMA in DMSO to 1 mM final concentration and incubating at 37° C. for 24 hours. The activated enzyme was diluted to 5 nM in TCNB assay buffer and 200 μL was added to each gel-containing well at the experiment start (t=0). At each time point, 2 μL supernatant was removed from each well and spectrophotometrically measured on the Nanodrop at 285 nm. An L-tryptophan standard curve was made in assay buffer with addition of the enzyme-activating molecule APMA (210 nM); this was used to calculate the concentration of cleaved peptide in the buffer over time for each gel. Between time points, the well plates were stored in the 37° C. incubator. The enzyme solution was refreshed every 36 hours by manually aspirating the remaining buffer from wells and replacing with newly activated enzyme in 200 μL buffer per well.

Cell Culture: RAW 264.7 cells were cultured in DMEM with 10% FBS and 1% penicillin/streptomycin at 37° C. and 5% CO₂. Depending on the experiment, cells were seeded in tissue culture polystyrene (TCPS) well plates (6, 24, or 95 well) or on hydrogels at 625,000 cells/cm² and allowed to adhere overnight. For transwell experiments, a transwell with a hydrogel was inserted into the well after the cells were allowed to adhere. For the (T6167923 (Br), T6167923 (SH)), and inhibitor-PEG-peptide studies, cells were treated with inhibitor for a specified period of time. To do this, medium was aspirated from the well and replaced with inhibitor-containing DMEM. Control conditions included cells only and cells plus 1% anhydrous DMSO by volume. After a specified period of culture time, PAM3CSK4 was added at 50 ng/ml and the cells were cultured for a prescribed period of time. Medium was collected and flash frozen for ELISA. For cell uptake studies, cells were cultured at 37° C. and 5% CO₂ in DMEM media containing 10% FBS and 1% penicillin/streptomycin, then seeded at 47,000 cells/cm² in 6-well TCPS plates. After adhering overnight, medium was refreshed and macrophages were treated with or without 1 μg/mL LPS in the same medium. After 30 minutes, cells were treated with or without 55 μg/mL microparticles for 24 hours. Cells were disassociated from the TCPS with Accumax, scraped, and fixed with 4% PFA for flow cytometry. For each experiment, a study design is depicted in the figures.

Hydrogel Implantation Studies: C57bl6/J female mice 6-8 weeks of age were purchased from Jackson Laboratory. Four materials (therapeutic or unmodified PEG hydrogels) were implanted in separate subcutaneous pockets of each mouse above the shoulders and hips. Incisions were closed with surgical staples. After seven days post-implantation, mice were euthanized by carbon dioxide inhalation then by cervical dislocation. The hydrogels and the surrounding tissue were explanted and processed for histological analysis. All animal protocols follow the NIH guidelines for care and use of laboratory animals and were approved by the Institutional Animal Care and Use Committee (IACUC) at the University of Colorado.

Cell Metabolic Activity: The metabolic activity of macrophages in vitro was determined with alamarBlue™ assay. Following treatment duration, medium was removed and replaced with 10% alamarBlue reagent in DMEM medium. After six hours of incubation at 37° C. and 5% CO₂ the absorbance was read at 570 and 600 nm. The metabolic activity was calculated per manufacturer instructions and normalized to the cells only positive control condition. The number of replicates was 3-4 per condition.

Protein Assays: Cell culture medium was thawed on ice. IL-6 in the culture media was quantified using an ELISA. MMP-12 production in culture media was measured per kit instructions. The number of replicates was n=4 for each condition.

Inflammatory Cell Layer Characterization: Explants from subcutaneous implantation studies were processed for paraffin embedding. Samples were sectioned into 5 μm thick slices, and stained with Masson's Trichrome; hematoxylin is used as a nuclear counterstain. Samples were imaged with brightfield microscopy (Axiovert 40C Zeiss). The layer of inflammatory cells at the surface of the hydrogels was defined by the distance of the layer of purple nuclei from the hydrogel surface to the collagenous fascia layer. Thickness was quantified using NIH ImageJ.

Statistical Analysis: The data analysis was generated using the Real Statistics Resource Pack software (Release 7.6). Copyright (2013-2021) Charles Zaiontz. www.real-statistics.com. The Shapiro-Wilks and Levene's tests were used to confirm if data were parametric. Parametric data were analyzed by one-way or two-way ANOVA and Tukey's post-hoc analysis. In the case of two-way ANOVA, data with significant interaction was subjected to one-way ANOVA or t-test. The Kruskal-Wallis test followed by Pairwise Mann-Whitney was performed on non-parametric data. Alpha, the confidence level, was 0.05. p<0.05 was considered statistically significant. Data are reported as means with standard deviations written parenthetically or plotted as error bars.

The invention addresses the need for improving the biocompatibility of implantable medical devices. While many medical devices can function with a fibrous capsule, this capsule is responsible for implant failure (e.g., implant loosening of joint arthroplasty) and has inhibited advancement of implantable sensors where sensitivity of the sensor depends on direct communication with the host.

The foreign body response (FBR) occurs essentially to all non-biological materials that are implanted into mammals. The FBR is characterized by chronic inflammation that leads to the walling-off of implants by a fibrous capsule, which can lead to implant failure. We have identified that Toll-like receptors (TLRs) mediate the FBR and that inhibiting, specifically TLR2 and TLR4 inhibits fibrous capsule formation to several different types of implants.

TLR2 and separately TLR4 either have no role or have a partial role in mitigating the FBR depending on the type of material. However, when both TLR2 and TLR4 are mutated in a mouse model, the fibrous capsule is substantially reduced across several different types of implants and to levels that are similar across the materials.

### Glossary of Claim Terms

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

The term “administration” and variants thereof (e.g., “administering” a compound) in reference to a compound of the invention means introducing the compound into the system of the subject in need of treatment.

As used herein, the term “composition” is intended to encompass a product comprising the specified ingredients in the specified amounts, as well as any product which results, directly or indirectly, from combination of the specified ingredients in the specified amounts.

As used herein, “treatment” refers to obtaining beneficial or desired clinical results. Beneficial or desired clinical results include, but are not limited to, any one or more of: alleviation of one or more symptoms (such as FBR), diminishment of extent of the formation of a fibrous capsule, stabilized (i.e., not worsening) state the formation of a fibrous capsule, preventing or delaying spread the formation of a fibrous capsule, preventing or delaying occurrence or recurrence or slowing of the formation of a fibrous capsule progression, amelioration of the state. The methods of the invention contemplate any one or more of these aspects of treatment.

A “pharmaceutically acceptable” component is one that is suitable for use with humans and/or animals without undue adverse side effects (such as toxicity, irritation, and allergic response) commensurate with a reasonable benefit/risk ratio.

A “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention.

A medical device is an instrument, tool, machine, test kit, or implant that is used to prevent, diagnose, or treat disease or other conditions. A medical device is defined as implantable if it is either partly or totally introduced, surgically or medically, into the human body and is intended to remain there after the procedure. As defined by the FDA, an implantable medical device is a device that is placed into the human body for a period of 30 days or more.

According to the National Cancer Institute, a small molecule is a drug that can enter cells easily because it has a low molecular weight. Once inside the cells, it can affect other molecules, such as proteins, and may cause cancer cells to die. This is different from drugs that have a large molecular weight, which keeps them from getting inside cells easily. Many targeted therapies are small-molecule drugs.

Encapsulation is the effective surrounding of a therapeutic agent that provides protection and/or release of the therapeutic agent. Encapsulation is an effective tool for targeted deliveries of drugs and sensitive compounds. The agent used for encapsulation is the encapsulating agent. Encapsulation approaches are based on barriers made from (bio) polymers, liposomes, multiple emulsions, etc.

Sustained release technology is a class of technology characterized by slowly-releasing specific active substances into a target medium to keep a certain concentration in the system within valid time. As used herein, the term includes extended-release and controlled release of a drug or dosage.

Biodegradable polymeric materials have been used for extended release. One such example is polylactic acid copolymer, which degrades to lactic acid and eliminates the problem of retrieval after implantation. Other polymers for drug formulations include polyacrylate, methacrylate, polyester, ethylene-vinyl acetate copolymer (EVA), polyglycolide, polylactide, and silicone. Of these, the hydrophilic polymers, such as polylactic acid and polyglycolic acid, erode in water and release the drug gradually over time. A hydrophobic polymer such as EVA releases the drug over a longer duration time of weeks or months. The rate of release may be controlled by blending two polymers and increasing the proportion of the more hydrophilic polymer, thus increasing the rate of drug release. [Chapter 17. Modified-Release Drug Products. In: Shargel L, Wu-Pong S, Yu A C. eds. Applied Biopharmaceutics & Pharmacokinetics, 6e. McGraw Hill; 2012.]

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

All references cited in the present application are incorporated in their entirety herein by reference to the extent not inconsistent herewith.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

TABLE 1
TLR4 Antagonists

M62812
TLR4-IN-C34
TAK 242-(R)-Ethyl 6-(N-(2-chloro-4-
fluorophenyl)sulfamoyl)cyclohex-1-enecarboxylate
Amitriptyline
Cyclobenzaprine
Eritoran
Ketotifen
Imipramine
Mianserin
Ibudilast
Pinocembrin
Resatorvid
M62812
Naloxone
(+)-Naloxone
Naltrexone
(+)-Naltrexone
LPS-RS
Propentofylline
Pentoxifylline
Tapentadol (mixed agonist/antagonist)
TLR4-IN-C34
Palmitoylethanolamide
T5342126/T5342126 hydrochloride
IAXO-102
Semapimod/Semapimod HCL

TABLE 2
TLR2 Antagonists

TLR2-IN-C29
MMG 11-Ethyl 5-(2-oxo-2-(2,3,4-
trihydroxyphenyl)ethyl)furan-2-carboxylate
CU CPT 22 - 3,4,6-Trihydroxy-2-methoxy-5-oxo-5H-
benzocycloheptene-8-carboxylic acid hexyl ester
SMU-Y6
Sparstolonin B
E567
o-vanillin (C29L, ortho-Vanillin)
SIG1459

TABLE 3
Small molecule and peptide inhibitors of MyD88

T6167923
YM155
ST2825
TJ-M2010-5
MyD88-IN-1
Compound 4210 (MyD88 inhibitor 4210)
T5910047
MyD88 Inhibitor 15d
MyD88 Inhibitor C17
TJ-M2010-2
Peptide inhibitor of the BB loop of MyD88
RDVLPGT [SEQ ID NO. 4] attached to a cell
penetrating peptide RQIKIWFQNRRMKWKK
[SEQ ID NO. 5]

TABLE 4
Small molecule and peptide inhibitors of TRIF
Resatorvid

Peptide inhibitor of the BB loop of
[SEQ ID NO. 6]
TRIF FCEEFQVPGRGELH
attached to a cell penetrating peptide
[SEQ ID NO. 7]
RQIKIWFQNRRMKWKK

| | |
|---|---|
| Compound 2* | |
| [symbol] | |
| 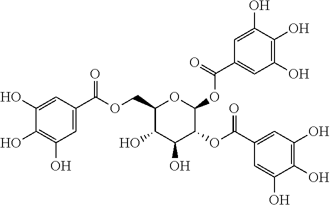 [SMILES: O=C(OC[C@H]1O[C@@H](OC(=O)c2cc(O)c(O)c(O)c2)[C@H](OC(=O)c2cc(O)c(O)c(O)c2)[C@@H](O)[C@@H]1O)c1cc(O)c(O)c(O)c1] | Compound 2 |
| Compound 4* | |
| [symbol] | |
| 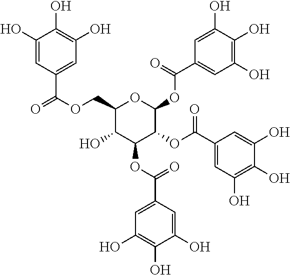 [SMILES: O=C(OC[C@H]1O[C@@H](OC(=O)c2cc(O)c(O)c(O)c2)[C@H](OC(=O)c2cc(O)c(O)c(O)c2)[C@@H](OC(=O)c2cc(O)c(O)c(O)c2)[C@@H]1O)c1cc(O)c(O)c(O)c1] | Compound 4 |

*Verma S, Reddy P, Sowdhamini R. Integrated approaches for the recognition of small molecule inhibitors for Toll-like receptor 4. Comput Struct Biotechnol J. 2023 Jul 22;21:3680-3689. doi:10.1016/j.csbj.2023.07.026. PMID: 37576745; PMCID: PMC1041283; see also Mahita J, Harini K, Rao Pichika M, Sowdhamini R. An in silico approach towards the identification of novel inhibitors of the TLR-4 signaling pathway. J Biomol Struct Dyn. 2016 Jun;34(6): 1345-62. doi: 10.1080/07391102.2015.1079243. Epub 2015 Sep 2. PMID: 26264972. [symbol]
indicates data missing or illegible when filed

TABLE 5
cFLIP/Survivin Inhibitors

| Inhibitor | Target Protein/Pathway |
|---|---|
| YM155 | Survivin at mRNA, protein, and transcription |
| | levels; cFLIP |
| FL118 | Survivin at mRNA and protein levels |
| SF002-96-1 | Survivin by inhibition of STAT3 and NF-kB |
| Terameprocol | Survivin |
| WM-127 | Survivin |
| GDP366 | Survivin at gene and protein levels |
| Abbot 8 | Survivin dimerization |
| LLP3 | Survivin dimerization |
| LLP9 | Survivin dimerization |
| S12 | Survivin dimerization |
| Indinavir | Survivin protein |
| Nelfinavir | Survivin protein |
| LQZ-7 | Survivin dissociation and degradation |
| LQZ-7F | Survivin dissociation and degradation |
| LQZ-7I | Survivin dissociation and degradation |
| Shepherdin | Survivin interactions with Hsp90 |
| AICAR | Sruvivin interactions with Hsp90 |
| Deazaflavin | Survivin interaction with Smac |
| analog compound | |
| 1 | |
| UC-112 | Survivin degradation through ubiquitin-mediated |
| | pathway |
| MX-106 | Survivin degradation through ubiquitin-mediated |
| | pathway |
| Compound 12b, | Survivin degradation through ubiquitin-mediated |
| 10f, 10h, 10k, 10n | pathway |
| PZ-6-QN | Survivin interaction with Smac |
| | Adapted from: Albadari N, Li W. Survivin |
| | Small Molecules Inhibitors: Recent Advances |
| | and Challenges. Molecules. 2023 Feb. 1; 28(3): |
| | 1376. doi: 10.3390/molecules28031376. |
| | PMID: 36771042; PMCID: PMC9919791. |

What is claimed is:

1. A coating for an implantable biomaterial comprising a small molecule TLR2 inhibitor conjugated to a first degradable linker and a small molecule TLR4 inhibitor conjugated to a second degradable linker, wherein the degradable linkers have a cleavage site that is cleavable by macrophage elastase and the linkers facilitate immobilization of the small molecule inhibitors on the surface of an implantable biomaterial.

2. The coating according to claim 1 further comprising a cFLIP inhibitor conjugated to a degradable linker, wherein the linker facilitates immobilization of the cFLIP inhibitor on the surface of an implantable biomaterial.

3. The coating according to claim 2 wherein the cFLIP inhibitor is YM155.

4. The coating according to claim 2 wherein the cFLIP inhibitor is a cFLIP inhibitor selected from Table 5.

5. The coating according to claim 1 further comprising an implantable biomaterial wherein the small molecule inhibitors with the linkers are releasably affixed to the surface of the implantable biomaterial.

6. (canceled)

7. The coating according to claim 1 wherein the TLR2 inhibitor is selected from the group consisting of MMG11 and TLR2-IN-C29.

8. The coating according to claim 1 wherein the TLR2 inhibitor is an inhibitor listed in Table 2.

9. The coating according to claim 1 wherein the TLR4 inhibitor is selected from the group consisting of TAK 242, TLR4-IN-C34, and M62812.

10. The coating according to claim 1 wherein the TLR4 inhibitor is an inhibitor listed in Table 1.

11. The coating according to claim 1 wherein the cleavage site that is cleavable by macrophage elastase comprises a sequence that is cleavable by an MMP12 enzyme.

12. The coating according to claim 11 wherein the cleavage site sequence is 70%, 80%, or 90% identical to SEQ ID NO: 1, SEQ ID NO: 2 or SEQ ID NO: 3.

13. The coating according to claim 11 wherein the cleavage site sequence is identical to SEQ ID NO: 1, SEQ ID NO: 2 or SEQ ID NO: 3.

14. The coating according to claim 1 wherein the linker is a peptide-tether that is sensitive to enzymes that catalyze the hydrolysis of peptidic bonds and the enzymes are overexpressed responsive to inflammation.

15. (canceled)

16. (canceled)

17. An implantable biomaterial having a surface of poly(ether ether ketone) (PEEK), medical grade silicone (MGS) or Poly(ethylene glycol) (PEG) and the surface coating according to claim 1.

18. A coating for an implantable biomaterial comprising a TLR2 inhibitor, a TLR4 inhibitor, and an encapsulating agent or hydrogel, wherein the inhibitors are conjugated to the encapsulating agent or hydrogel and the encapsulating agent or hydrogel is capable of immobilization on the surface of an implantable medical device.

19. The coating according to claim 18 wherein the TLR2 inhibitor and the TLR4 inhibitor are conjugated to the encapsulating agent.

20. The coating according to claim 18 wherein the TLR2 inhibitor is selected from the group consisting of MMG11 and TLR2-IN-C29.

21. (canceled)

22. The coating according to claim 18 wherein the TLR4 inhibitor is selected from the group consisting of TAK 242, TLR4-IN-C34, and M62812.

23. (canceled)

24. The coating according to claim 18 further comprising a cFLIP inhibitor.

25. The coating according to claim 24 wherein the cFLIP inhibitor is YM155 and the YM155 is conjugated to the encapsulating agent.

26-71. (canceled)
